(19) United States
(12) Patent Application Publication
(10) Pub. No.: US 2024/0238053 A1
(43) Pub. Date: Jul. 18, 2024

(54) DEVICE AND METHOD FOR TREATMENT OF DEEP VEIN THROMBOSIS AND PULMONARY EMBOLISM

(71) Applicant: Mark A. Litman, Edina, MN (US)

(72) Inventor: Mark A. Litman, Edina, MN (US)

(21) Appl. No.: 18/401,404

(22) Filed: Dec. 30, 2023

Related U.S. Application Data
(63) Continuation-in-part of application No. 17/460,210, filed on Aug. 28, 2021.

(51) Int. Cl. A61B 34/00; A61B 17/00; A61B 17/221; A61B 34/30; A61B 90/00; G16H 20/40

(52) U.S. Cl. CPC A61B 34/25; A61B 17/221; G16H 20/40; A61B 2017/00119; A61B 2034/252; A61B 2034/303; A61B 2090/374; A61B 2090/378

(57) ABSTRACT

A method of alerting a user to the existence of an anomalous variation in local conditions during use of a thrombus retrieval device in a patient. The method including: inserting a thrombus retrieval device into a blood vessel in a patient having a thrombus within the vessel while a region including the thrombus is under sensed observation. Sensing a signal responsive to at least thrombus variations, the variations being in response to the use of the thrombus retrieval device, sensing the existence of the anomalous variation in the local conditions, with a server receiving data of the sensed anomalous variation in local conditions. The server executes software to identify available procedures to be used after identification of one or more anomalous variations in local conditions related to the sensed anomalous variation in local conditions. The server provides first visual data visible to a human eye identifying the available procedures.

### BACKGROUND OF THE INVENTION

### 1. Field of the Invention

This invention pertains to intravascular medical devices for isolating, capturing, and removing blood clots from a blood vessel. This same system may also be used to retrieve obstructions, using coils, balloons, or catheter fragments dislodged during interventional procedures from the blood stream. The same system may also be used to remove obstructions from ducts and other cavities of the body, such as, for example, foreign bodies or stones from the urinary or the biliary tracts. In particular, this invention relates to medical devices for the intravascular treatment of deep vein thrombosis (DVT) and acute pulmonary embolism (PE). More particularly, this invention relates to the use of Artificial Intelligence in combination with thrombectomy devices to improve the function of the devices during live surgical procedures.

### 2. Background of the Art

The present invention pertains generally to thrombus that may produce a clot in a patient's vasculature. Clots can restrict blood flow to body tissues, in which blockage or obstruction may lead to serious medical consequences, including DVT and PE. Thromboembolism occurs when a blood clot trapped within a blood vessel breaks loose and travels through the blood stream to another location in the circulatory system, resulting in an obstruction at the new location. When a clot forms in the venous circulation, it may lodge within a pulmonary blood vessel causing PE. A PE can decrease blood flow through the lungs, which in turn causes decreased oxygenation of the lungs, heart and rest of the body.

Conventional approaches to treating thromboembolism include clot reduction and/or removal. Anticoagulants can prevent additional clots from forming and thrombolytics can be partially disintegrate the clot. However, such agents typically take a prolonged period of time and in some instances can induce hemorrhage. Transcatheter clot devices can cause trauma to the vessel, are hard to navigate to the pulmonary embolism site, and may be expensive to manufacture. Surgical procedures come with increased cost, procedure time, risk of infection, higher morbidity, higher mortality, and recovery time. Accordingly, there is need for better devices and methods.

DVT and PE are considered as part of the same venous thromboembolism (VTE) disease process. The most frequent long-term complication of DVT is post-thrombotic syndrome (PTS). Veins in the leg or pelvis are most commonly affected, including the popliteal vein, femoral vein, iliac veins of the pelvis, and the inferior vena cava. Upper extremity DVT most commonly affects the subclavian, axillary, and jugular veins.

Acute PE represents the most serious clinical manifestation of VTE disease. In patients with hemodynamically significant PE, systemic thrombolysis improves right ventricular dysfunction and reduces pulmonary artery pressures. However, systemic thrombolysis is associated with a risk of bleeding, particularly intracranial hemorrhage. An alternative to direct infusion into the pulmonary artery using an infusion catheter may provide the benefit of clot retraction to reduce the risk of bleeding.

Once DVT or PE has been diagnosed, treatments can range from anticoagulation alone, catheter-directed thrombolysis, full-dose systemic thrombolysis, reduced-dose systemic thrombolysis, catheter embolectomy, or surgical embolectomy. Anticoagulants can prevent additional clots from forming, and thrombolytics can dissolve the clot. However, such agents can cause hemorrhage and typically take hours or days before the treatment is effective.

Various medical devices have been used commercially in treating DVT and PE, including examples disclosed by U.S. Pat. Nos. 10,238,406, 10,524,811, 10,342,571, 10,098,651, 10,045,790, 10,588,655, 10,349,690, 10,335,186, 10,231,751, 9,844,387, 9,700,332, 9,408,620, 9,717,519, 9,439,664, and 9,427,252.

However, none of the devices currently available is ideal for treating DVT or PE. The ideal thrombectomy device would be designed to retract hard and soft clots in DVT and PE patients in a single pass without trauma to the vessel An essential aspect of a DVT/PE thrombectomy device is its effectiveness at removing obstructive thrombi, thereby achieving a rapid improvement in hemodynamics and avoiding ischemic complications. The ideal device would allow rapid passage and advancement into veins and arteries, but must also filter distal thrombi. The device must be safe for the patient without causing damage to vascular structures, and blood loss during the procedure must be minimized. Only 3-5% of DVT and PE cases are treated today with mechanical clot removal devices. Currently, all devices for thrombectomy are costly. There is therefore an ongoing unmet need for new devices and approaches that can safely and reliably removal clots in DVT and PE patients.

### SUMMARY OF THE INVENTION

A method alerts a user (technician, medical practitioner, nurse, robotics operator, etc.) to the existence of an anomalous variation in local conditions during insertion, deployment, manipulation or withdrawal of a thrombus capture/retrieval device in a subject (patient, human, mammal or lesser animal). The method may at least include: inserting a thrombus retrieval device into a blood vessel in a patient having a thrombus within the vessel while a region surrounding and including the thrombus is under sensed observation; sensing an output signal or visualization responsive to at least a size change, thrombus orientation change, or motion of or within or on the outside of the thrombus. The motion may be in response to the insertion, deployment, manipulation or withdrawal of the thrombus retrieval device; sensing the existence of the anomalous variation in the local conditions; a server receiving data of the sensed anomalous variation in local conditions; the server executing software to identify available procedures to be used after identification of one or more anomalous variations in local conditions; the server providing a first visual data visible (e.g., to a human eye of the user) identifying the available procedures, with or without a hierarchal preference among the available procedures; a medical practitioner manually executing at least one of the identified medical procedures or directing a robotic element to perform at least a portion of the identified medical procedure; and while undergoing continued sensed observation, additional sensed output is transmitted to the server, and effects of the execution of the available procedure are compared from the continued sensed observation to evaluate resultant effects on the anomalous variation in local conditions to determine what alteration has occurred in the anomalous variation in local conditions, identifying operational value (improvement, neutral results, deterioration) in the execution of the available procedure; and the server providing a second provided visual data identifying procedure status.

Artificial Intelligence may be used in combination with all intravascular thrombus retraction devices to improve surgical outcomes by addressing live time issues during surgery that can impact results. A preferred thrombectomy retrieval device includes wires that are compressible into a compact cylindrical form within a catheter and which are self-expandable into a wire mesh web with at least some parallel wires forming openings in the wire mesh sufficient to allow fluid passage and small enough to filter particles of at least 0.001 mm, a base of the wire mesh web connected to radial ring-shaped structure supporting and maintaining an opening in the base of the wire mesh and forming a thrombus capture volume, the radial ring-shaped structure being compressible into the catheter and being self-expandable when free of compressive forces within the catheter to open up into an open, expanded, radial ring-shaped structure which maintains the opening in the opening in the base of the wire mesh. There are also multiple intermediate guide wires connected to and spaced about the ring-shaped structure, the multiple intermediate guide wires are connected to withdrawal guidewires extending into the catheter. It is preferred that at least some of the wires in the wire mesh have protrusions extending inwardly into the thrombus capture volume, at least some of the protrusions having a height less than a distance between the at least some parallel wires.

An example of a method for performing a thrombectomy with a preferred device is also disclosed in which the method is executed by:
a) inserting the device into a blood vessel having a thrombus;
b) advancing a distal end of the catheter of the device towards a thrombus;
c) deploying the compressed wire mesh and ring-shaped structure distally past the thrombus;
d) expanding the wire mesh and ring-shaped structure past the thrombus;
e) retracting the wire mesh and ring-shaped structure by applying tension to the withdrawal guidewires in a first retraction step; and
f) capturing the thrombus within the wire mesh during the first retraction step.
   Cardiovascular disease may arise from accumulation of atheromatous material on the inner walls of vascular lumens. If a partially or completely occluded vessel provides blood to sensitive tissue such as the brain or heart, for example, serious tissue damage may result. Vascular deposits may restrict blood flow through an artery and can cause ischemia in the heart, legs, lungs, or the brain, which may lead to pain, swelling, wounds that will not heal, amputation, stroke, myocardial infarction, and/or other conditions.

The present disclosure discloses a medical device capable of retracting DVT and PE clots from blood vessels using a collecting mechanism and aspiration, so that the retraction device and clot are withdrawn proximally through the guiding catheter out of the body. Deposits may be treated by drugs, bypass surgery and atherectomy, including a variety of catheter-based approaches based on intravascular removal of deposits occluding a blood vessel. A catheter-based system may be utilized for removing a thrombus, wherein the catheter may be extended distal to a thrombus in a blood vessel wherein the thrombus is retracted from the vessel.

A limiting factor with available thrombectomy catheter devices is the difficulty to identify and treat hard thrombus. Current thrombectomy devices do not reliably break the thrombus away from the wall of the vessel. Current thrombectomy catheters are typically bulky and require manipulation towards the thrombus to avoid the risk of distal embolism.

Basic aspiration catheters have a proximal end connected to a suction pump which causes fluid to enter the distal opening of the hollow lumen and travel to the proximal end of the lumen. Conventional aspiration catheters are typically threaded through a balloon guide catheter. In one exemplary procedure, the balloon of the guide catheter is inflated to occlude the vessel. The distal end of the aspiration catheter is typically advanced to the blood clot, with suction connected to the aspiration catheter to cause flow reversal.

One fundamental issue with thrombectomy catheters is that thrombotic burden can be highly variable. Mechanical catheters may have size constraints with respect to their use on larger thrombi. Aspiration devices have operational limits when the diameter of the catheter limits their use to small thrombi. Large thrombi on the other hand, will not pass into the catheter, which creates a risk of embolism. Since blood is extracted alongside the thrombus in the thrombectomy procedure, aspiration can potentially cause hemodynamic deterioration in patients with pulmonary-embolism-related shock. The flexibility and durability of aspiration catheter systems may thus limit their use.

Thrombi normally must deform to the inner diameter of the aspiration catheter. The applied vacuum may partially draw a thrombus into the distal opening of the aspiration catheter's lumen, thereby deforming some of the thrombus to the catheter's inner diameter. If the thrombus becomes lodged within the distal opening of the aspiration catheter, the only option is to pull the clot back through the balloon guide. Pieces of the clot can break off during movement. When the clot is drawn out from the patient, it is difficult to confirm that the entire thrombus was removed.

An aspiration system that increases the first-pass recanalization rate can be a useful metric. Prior art systems are often not able to react quickly enough to keep the distal end of the catheter from experiencing a positive pressure. Thus, a need exists to overcome the problems with recanalization systems, designs, and processes.

One aspect of the present disclosure is to provide a mechanical thrombectomy system that is flexible enough so that it can reliably and safely navigate blood vessels to a clot.

A second aspect of the present disclosure is to provide a mechanical thrombectomy device that can reliably entrap a soft or hard thrombus without fragmenting the thrombus or damaging the intima of the blood vessel.

A third aspect of this disclosure is to provide a mechanical thrombectomy device that is biocompatible and compatible with standard medical catheters.

A fourth aspect of this disclosure is to provide a mechanical thrombectomy device that can safely and completely remove large clots of any density from the upper leg, pelvis, and lung.

A fifth aspect of the disclosure is to provide a mechanical thrombectomy device that reduces the risk of fragmentation and distal embolization when used in association with aspiration.

A sixth aspect of this disclosure is to provide an aspiration system that increases the first-pass recanalization rate during thrombus removal.

### BRIEF DESCRIPTION OF THE DRAWINGS

This invention may be more completely understood with respect to the following description of various embodiments. While the disclosure is amenable to various modifications and alternative forms, specifics have been shown by way of example in the drawings and will be described in detail.

FIG. 1 A-G show serial views of the intravascular thrombectomy catheter during isolating, capturing, and removing blood clots from a blood vessel.

FIG. 1A shows a schematic view of the catheter device for retracting intravascular thrombotic material.

FIG. 1B shows the intravascular thrombectomy catheter device with its distal tip extending beyond the distal edge of the thrombus.

FIG. 1C shows the thrombectomy catheter device collapsed inside the retraction catheter with the guidewire removed.

FIG. 1D shows the thrombectomy device with catheter components deployed.

FIG. 1E shows the retraction catheter being pulled with the thrombectomy device over the thrombus.

FIG. 1F shows the collecting basket being positioned to collect the thrombus.

FIG. 1G shows the collecting basket deployed over the thrombus.

FIG. 1H is a cross-sectional illustration of a multi-lumen catheter with an inserted optical fiber that can be used to measure an optical signal in a thrombus in an artery.

FIG. 1I is a schematic of the brain of a patient illustrating a thrombus retraction procedure in which fluoroscopic images may be acquired and stored in memory by an MRI-based tracking system.

FIG. 2A shows screen shots of received information and stored information and underlying algorithms stored on a central computer and instructions that may be provided in response to specific sensed input.

FIG. 2B shows sensed sources of information that may me used solely or in combination to identify the properties of specific clots, which identified properties can be input as in FIG. 2A to identify procedures.

FIG. 3 is a schematic view of the distal embolic protection component of the thrombectomy device.

FIG. 3A summarizes prior art studies in experimental animals.

FIG. 3B shows generic images of a vein 310 and an artery 320.

FIG. 4 is a schematic of a tracking system to evaluate DVT and PE treatment in individual patients.

FIG. 4A shows a modified clot classification system based on imaging and clot morphology.

FIG. 4B shows a modified clot classification system based on imaging and clot morphology.

FIG. 5 is a schematic view of the major veins in the upper leg, pelvis and thorax involved in DVT and PE.

FIG. 5B is a schematic of an MRI-based tracking system.

FIG. 6 is a schematic view of a custom-made handle to advance of the DVT/PE thrombectomy device.

FIG. 7 show the components of an aspiration device that can be added to the manifold attached to the hub of the guiding catheter or the collecting catheter.

FIG. 8 shows a mesh web wire having different protruding elements. protrusions, dots, or gripping elements that are on the wire, generally facing inward (towards where a clot or the thrombus would be in contact with the wire.

FIG. 9 shows a predeployed 9A catheter and forward compressed wire collection element.

FIG. 9B shows the deployed 9A catheter.

FIG. 10 shows a fully deployed capture system.

FIG. 10 A shows additional features added to the deployed system of FIG. 10.

FIG. 11 shows three different aspects or perspectives of the deployed ring-shaped structure's four segments.

FIG. 11A shows a first aspect or perspective of the deployed ring-shaped structure's four segments.

FIG. 11B shows a second aspect or perspective of the deployed ring-shaped structure's four segments.

FIG. 11C shows a third aspect or perspective of the deployed ring-shaped structure's four segments.

FIG. 12 shows a different perspective of the deployed ring-shaped structure segments of FIG. 11.

FIG. 13 shows a pre-deployed 13A catheter delivery system and deployed 13B catheter delivery assembly comprising a catheter, single retraction guidewire and multiple dual wire element distal retraction guidewires.

### DETAILED DESCRIPTION OF THE INVENTION

This disclosure provides design, material, manufacturing method, and use alternatives for medical devices and systems. The device for treating DVT and PE comprises accessing a venous blood vessel of a patient in which a retraction catheter is inserted to a site of clot. An aspiration catheter with wall-mounted suction may be attached to remove a vascular obstruction with one pass.

Aspiration may be applied to the guiding or collecting catheters to decrease embolization of clot fragments.

An intravascular thrombus retraction device includes wires that are compressible into a compact cylindrical form within a catheter and which are self-expandable into a wire mesh web with at least some parallel wires forming openings in the wire mesh sufficient to allow fluid passage and small enough to filter particles of at least 0.001 mm, a base of the wire mesh web connected to radial ring-shaped structure supporting and maintaining an opening in the base of the wire mesh and forming a thrombus capture volume, the radial ring-shaped structure being compressible into the catheter and being self-expandable when free of compressive forces within the catheter to open up into an open, expanded, radial ring-shaped structure which maintains the opening in the opening in the base of the wire mesh. There are also multiple intermediate guide wires connected to and spaced about the ring-shaped structure, the multiple intermediate guide wires are connected to withdrawal guidewires extending into the catheter. It is preferred that at least some of the wires in the wire mesh have protrusions extending inwardly into the thrombus capture volume, at least some of the protrusions having a height less than a distance between the at least some parallel wires.

An alternative intravascular thrombus retraction device includes wires that are compressible into a compact cylindrical form within a catheter and which are self-expandable into a wire mesh web with at least some parallel or helical wires forming mesh openings in the wire mesh sufficient to allow aqueous fluid passage and small enough to filter particles of at least 0.001 mm or thrombus particles having a size which is recognized as having potentially harmful effects in at least the smaller blood vessels in the brain. A base of the wire mesh web is connected to radial ring-shaped structure supporting and maintaining an opening in the base of the wire mesh and forming a thrombus capture volume within the wire mesh. The radial ring-shaped structure could in theory be a single continuous element having an elastic memory that is the radial ring shape, but for purposes of construction of the device, a radial ring element having more than two bend or flex points, of having pivots, rotating connections, or segmented elements that allow for easier and more shapely compression may be used. The radial ring-shaped structure is as described compressible into a thin roughly cylindrical shape within the catheter and is self-expandable when free of compressive forces within the catheter to open up into an open, expanded, radial ring-shaped structure which maintains the opening in the opening in the base of the wire mesh. There are also multiple intermediate guide wires connected to and spaced about the ring-shaped structure, the multiple intermediate guide wires are connected to withdrawal guidewires extending into the catheter. It is preferred that at least some of the wires in the wire mesh have protrusions extending inwardly into the thrombus capture volume, at least some of the protrusions having a height less than a distance between the at least some parallel wires.

The wire may be a non-thrombogenic metal and the protrusions have a height of less than 0.001 mm. Because of the relatively short time duration of the device within a blood stream, there may be a tolerable range of materials that can be used if they are non-thrombogenic within the time frame of the surgery. The protrusions may be elements, bumps, rods, and the like extending from surfaces of the wires and the protrusions may have concave, convex, flat, curvilinear or pointed tips.

The device may include two catheters, a first catheter containing the wire mesh and ring-shaped structure in a compressed, non-expanded state, and a second catheter containing a compressed and expandable collection receptacle, the collection receptacle positioned within the second catheter such that upon release from the catheter, the collection receptacle expands to provide an opening in an opposed position with respect to the opening in the base of the wire mesh of a released and expanded wire mesh and ring-shaped structure.

The device may have the ring-shaped structure include or be attached to struts which place expanding or restraining force on the ring-shaped structure to maintain the opening in an expanded and open position.

The device may also or alternatively have the collection receptacle include or be attached to struts which place expanding or restraining force on the opening in the opposed position to maintain the opening in the opposed position in an expanded and open position.

A method of capturing a thrombus within vasculature may include comprising providing the above described intravascular thrombus retraction device, which may alternatively be characterized as wires that are compressible into a compact cylindrical form within a catheter and which are self-expandable into a wire mesh web with at least some parallel wires forming openings in the wire mesh sufficient to allow fluid passage and small enough to filter particles of at least 0.001 mm, a base of the wire mesh web connected to radially ring-shaped structure supporting and maintaining an opening in the base of the wire mesh and forming a thrombus capture volume, the ring-shaped structure being compressible into the catheter and being self-expandable when free of compressive forces within the catheter to open up into the open, expanded ring-shaped structure, maintaining the opening in the opening in the base of the wire mesh, multiple intermediate guide wires are connected to and spaced about the ring-shaped structure, the multiple intermediate guide wires are connected to withdrawal guidewires extending into the catheter, at least some of the wires in the wire mesh having protrusions extending inwardly into the thrombus capture volume, at least some of the protrusions having a height less than a distance between the at least some parallel wires; the method comprising:
a) inserting the device into a blood vessel having a thrombus;
b) advancing a distal end of the catheter of the device towards a thrombus;
c) deploying the compressed wire mesh and ring-shaped structure distally past the thrombus;
d) expanding the wire mesh and ring-shaped structure past the thrombus;
e) retracting the wire mesh and ring-shaped structure by applying tension to the withdrawal guidewires in a first retraction step; and
f) capturing the thrombus within the wire mesh during the first retraction step.

The wire may be composed of a non-thrombogenic metal and the protrusions have a height of less than 0.001 mm, and during the first retraction step, the protrusions engage and grasp a surface of the thrombus.

The present disclosure further relates to a method of treating DVT and PE in the peripheral vasculature of a patient. The method includes providing a thrombectomy device that can be tubular and is formed of a braided filament mesh structure. The mesh structure can have a proximal end of the attached to a distal end. The invention includes advancing a catheter with the thrombectomy device through a vascular thrombus in a venous vessel. A shaft extends through the catheter and a distal end is coupled to a proximal end. The method includes deploying the thrombectomy device from the catheter from a constrained configuration to an expanded configuration. In some embodiments, the thrombectomy device engages at least a wall of the venous vessel distally past the thrombus at full expansion. The method includes retracting the thrombectomy device proximally to separate a portion of the thrombus from the venous vessel wall while the mesh structure captures the thrombus. The method includes withdrawing the thrombectomy device from the patient to remove the thrombus from the venous vessel.

Advancing the thrombectomy device includes inserting the catheter into the venous vessel until a radiopaque distal tip of the catheter is distally past the thrombus. In some embodiments, deploying the thrombectomy device from the constrained configuration to the expanded configuration includes advancing the shaft distally until the thrombectomy device is beyond a distal end of the catheter. Deploying the thrombectomy device further includes determining a position of the thrombectomy device with respect to the catheter via imaging of a first radiopaque marker located on the catheter and a second radiopaque marker located on at least one of the shaft or mesh structure.

The vascular thrombectomy device is added into the mesh structure by entering the expandable tubular portion via at least an aperture located at the proximal end of the self-expanding stent. The method includes inserting the catheter into the venous vessel through an access site, which is a popliteal venous site, a femoral venous site, or an internal jugular venous site. The venous vessel has a diameter of at least 5 millimeters and may include a femoral vein, an iliac vein, a popliteal vein, a posterior tibial vein, an anterior tibial vein, or a peroneal vein.

The method further includes: percutaneously accessing the venous vessel of the patient with an introducer sheath through an access site into the venous vessel of the patient; advancing a distal end of the introducer sheath to a position proximal of the thrombus; inserting the catheter through a lumen of the introducer sheath so that a distal tip of the catheter is distally past the thrombus.

Withdrawing the thrombectomy device from the patient includes: retracting the thrombus extraction device relative to the introducer sheath until an opening is within the self-expanding stent; collapsing the stent portion and mesh structure to compress the thrombus; retracting the stent portion and mesh structure into the introducer sheath; and removing the thrombectomy device from the introducer sheath.

The method may further include extruding at least some of the thrombus through the distal portion of the expandable tubular portion and capturing a part of the thrombus in the self-expanding funnel or further compressing the thrombus through a mesh of the self-expanding funnel. The method may further includes aspirating the thrombus through an aspiration port connected to a proximal end of the introducer sheath.

One aspect of the present disclosure relates to a method of treating DVT in a peripheral vasculature of a patient to include percutaneously accessing a venous vessel of a patient with an introducer sheath through a popliteal vein site; and inserting a catheter with a thrombectomy device through a lumen of the introducer sheath so that the catheter is distally past the thrombus.

In some embodiments of invention, a proximal end of the mesh structure may be attached to a distal end of the fenestrated structure. The thrombectomy device may be deployed from a constrained configuration to an expanded configuration by advancing a shaft distally until the stent portion of the thrombectomy device is beyond the distal end of the catheter.

One aspect of the present invention relates to a removal of thrombus from an artery or a vein of a patient by providing a thrombectomy device with a net-like filament mesh structure; advancing with the thrombectomy device through a thrombus, and deploying the thrombectomy device to engage a wall of the blood vessel. Retracting the thrombectomy device to separate a portion of the thrombus from the vessel wall and to capture the portion of the thrombus within the net-like mesh structure to remove thrombus from the patient.

In the method of the invention, fluoroscopically monitoring deployment of the thrombectomy device beyond first radiopaque marker located on the catheter relative to a second radiopaque marker located on the thrombectomy device. In some embodiments, the thrombus is located in the peripheral vasculature of the patient and the blood vessel has a diameter of at least 5 millimeters and includes at least one of a femoral vein, an iliac vein, a popliteal vein, a posterior tibial vein, an anterior tibial vein, or a peroneal vein. In some embodiments of the invention, the method includes aspirating or infusing a thrombolytic agent into or from the blood vessel before, during, or after thrombus extraction.

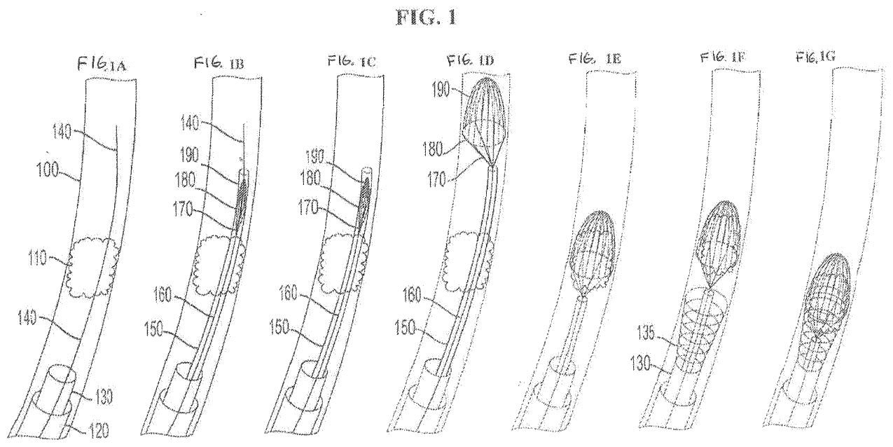

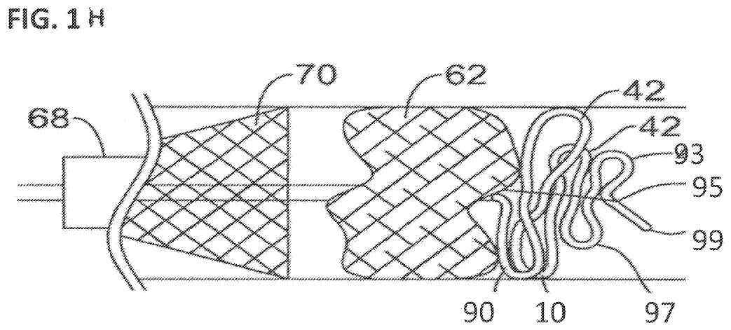

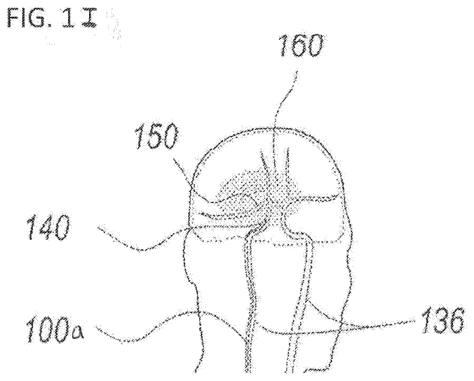

FIGS. 1 A-G depict steps for the mechanical thrombectomy device in a blood vessel 1 with a thrombus 2. A guiding catheter 3 can be positioned by transluminal catheter delivery within the lumen of the blood vessel 1 proximal to the thrombus using image-guided techniques. A collecting catheter can pass through the guiding catheter and may be positioned just below the proximal aspect of clot 2. As shown in FIG. 1H, a guidewire 5 can be placed proximal or distal to the thrombus 2 to be used to guide the retraction catheter. The retraction catheter 6 can then pass through collecting catheter 4 and over guidewire 5 to a position with its distal tip placed distal to the distal edge of the thrombus 2 as shown in FIG. 1B. The thrombectomy device with retracting wire 7, struts 8, ring structure 9 and web 10 can be passed through the retraction catheter or may be pre-loaded inside the retraction catheter 6. As shown in FIG. 1C, the guidewire 5 can be removed. The thrombectomy device can be deployed by manipulating the retraction wire 7 as shown in FIG. 1D. As shown in FIG. 1E, the retraction catheter 6 can be positioned proximally with the thrombectomy device pulled down over thrombus 2. As shown in FIG. 1F, the collecting basket 11 may be deployed from the collecting catheter 4 to collect the thrombus as shown in FIG. 1G.

When possible, the entire thrombus may be pulled into the guiding catheter 3 and removed from the body, leaving the guiding catheter 3 in place. If the clot is too large to be pulled into and through the guiding catheter 3, aspiration may be applied to the guiding catheter or the collecting catheter 4, wherein a catheter connected to an aspiration system can be hooked to the flushing system for the guiding catheter via a 3-way stopcock. Aspiration can be usefully added when applied to the clot that has been pulled into the collecting device 4 to make it smaller for removal through the guiding catheter 3.

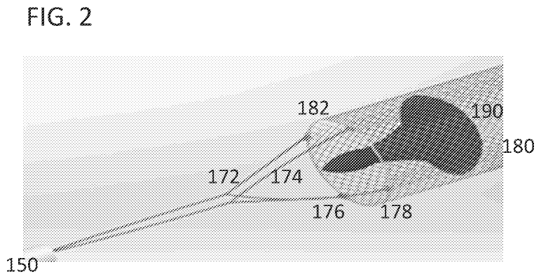

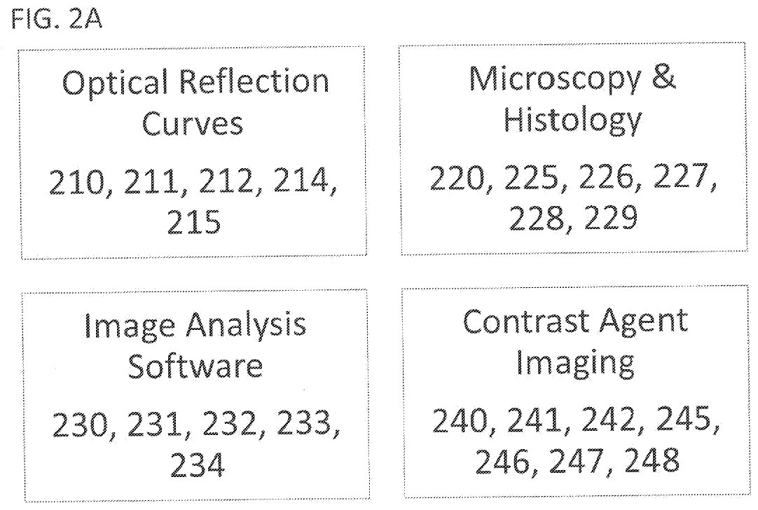

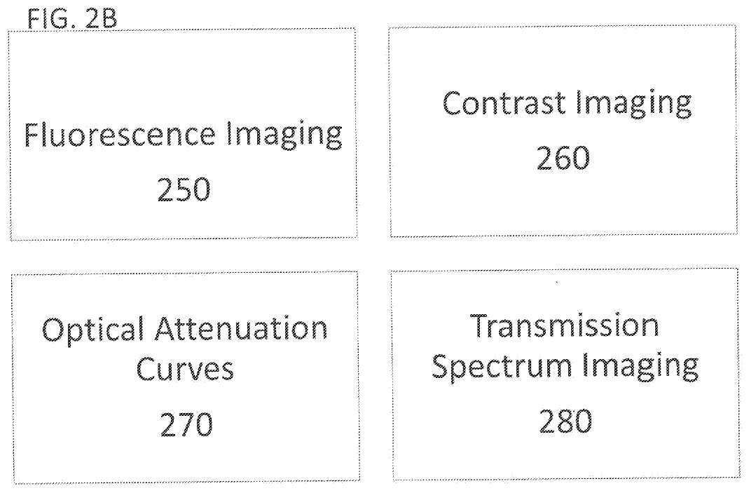

FIG. 2 is a schematic view of the thrombectomy device showing the arrangement of the struts 172, 174, 176, 178 and the ring segment 182. The device uses a web structure with retraction wires to retract thrombus 190. The device includes expandable struts having a closed compact configuration 184 and an open expanded configuration 186. In an optional embodiment, additional collection features such as cactus-claws, end-hooks and hook-type imaging may be included. In some embodiments, these features can be formed from various metals or alloys such as Nitinol, platinum, cobalt-chrome alloys, 35N LT, Elgiloy™, stainless steel, tungsten or titanium.

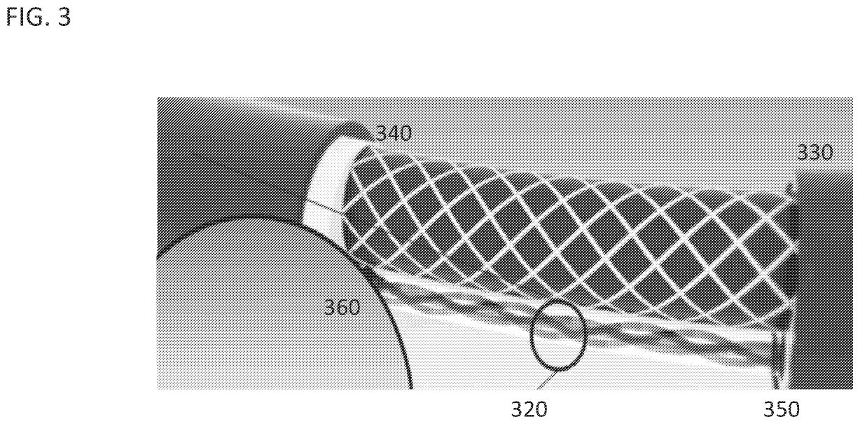

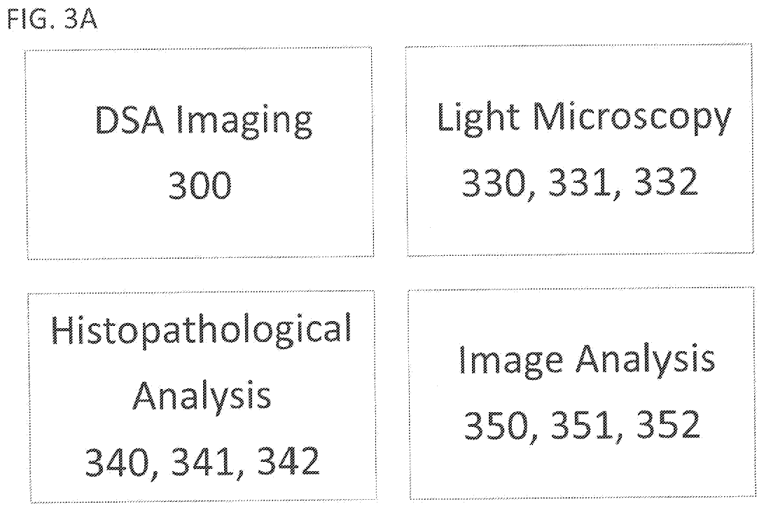

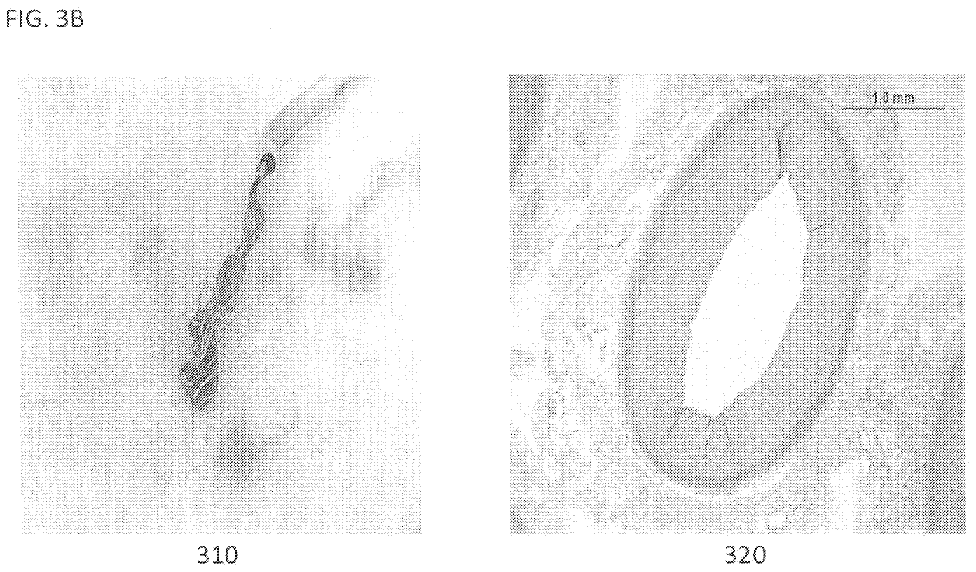

In one embodiment of the disclosure shown in FIG. 3, fluoroscopically visible markers 188 are applied to the retraction ring, the retraction wire and the collecting basket 180 and to the tip of the guiding catheter 150 to facilitate localization of all components. Some examples of radiopaque materials include gold, platinum, palladium, tantalum, tungsten alloy, and polymer material loaded with a radiopaque filler. The thrombectomy device may also be made from a metal, metal alloy, polymer, a metal-polymer composite, ceramics, or other suitable material including, such as304V, 304L, and 316LV stainless steel; mild steel; nickel-titanium alloy such as linear-elastic and/or super-elastic nitinol; and nickel-chromium-molybdenum alloys.

In another embodiment, the device can be equipped with imaging sensors 350, or with sensors measuring physiological parameters 360 such as pressure, temperature and oximetry. In one embodiment, FOSS technology facilitates the visualization of thrombectomy catheters 330 and wires 340 without the need for fluoroscopy. In addition to reducing the need for X-ray exposure of the patient and medical personnel, the FOSS technology also enables more detailed views of device positioning. In an exemplary embodiment, optical fibers are embedded in the device and equipped with Fiber Bragg Gratings, which enables the determination in 3 dimensions of the shape and position of the catheters and wires in real-time and with high accuracy. The shape and position of the catheters and wires can then be superimposed on roadmap views of the vasculature and pathology.

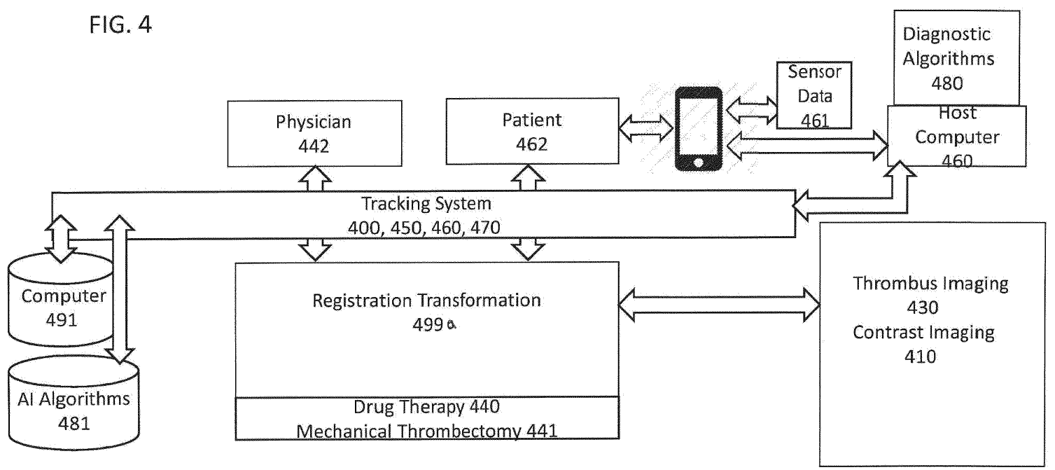

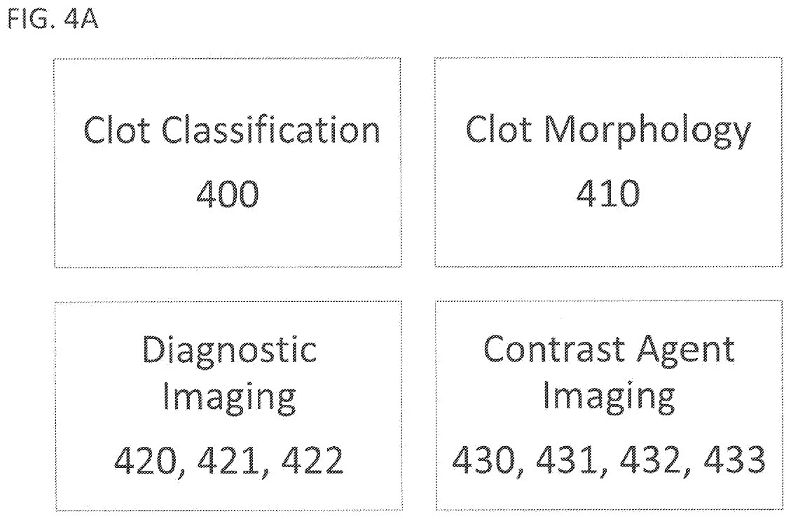

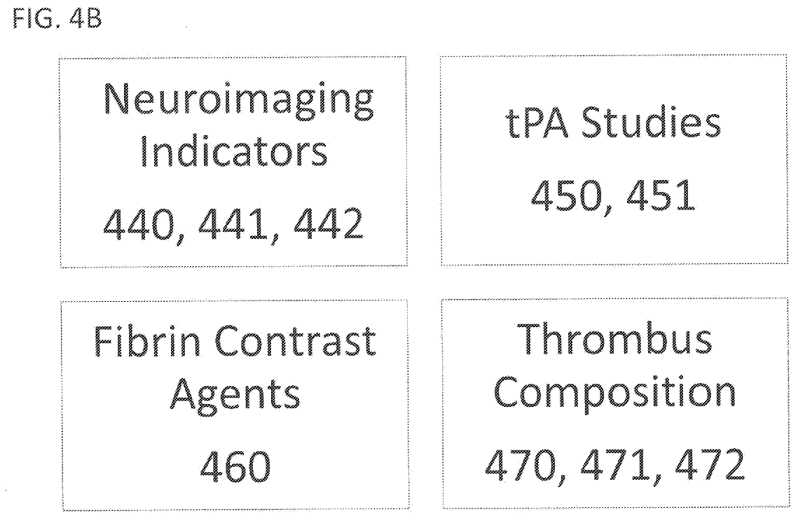

As shown in FIG. 4, sensors 461 may be connected to a host computer 461, wherein diagnostic algorithms 480 can be used to evaluate treatment plans for individual patients 462 and to actively modify existing treatment plans by physicians 442. A computer-based 491 tracking system 400 can be used in DVT and PE patient studies to investigate clot composition with respect to the age 450, composition 460 and size of a thrombus 470. During formation of a thrombus, characteristic alterations in fluorescence contrast imaging 410 and thrombus imaging 430 may be registered by the host computer 499. Observation of imaging changes 499 can be clinically useful in evaluating the potential utility of various alternative therapeutic interventions, such as, for example, drug thrombolytic therapy 440 and mechanical thrombectomy 441. Imaging 410, 430 may be used to guide the thrombectomy device through the vasculature 510 through under manual control of a host computer 460. Since branching of the main pulmonary arteries may be anatomically complex, it may be difficult to locate and catheterize an occluded vessel 499 when using single plane fluoroscopy 410.

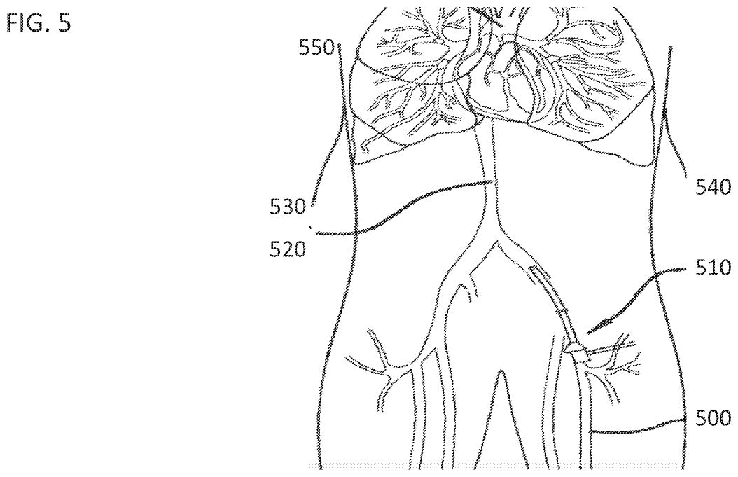

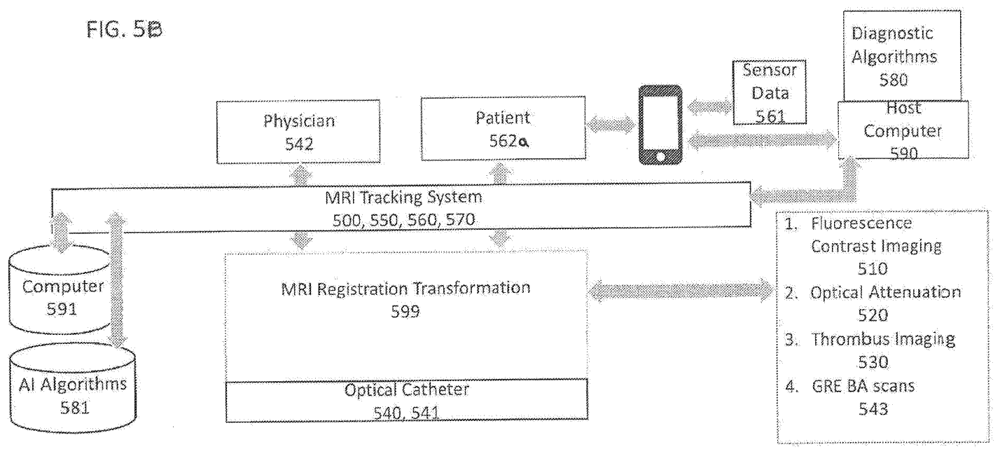

As shown in FIG. 5B, image guidance can be used to guide the thrombectomy device through the vasculature 500 toward the correct location 530, 540, to manipulate various functions of the device 510, and to manage the retraction of the thrombus 520. PE is considered to be part of the same continuum of disease as DVT with over 95% of emboli originating in the legs. Mechanical clot removal, as disclosed in the present disclosure, is relatively rapid compared to the use of thrombolytics. Pulmonary emboli, particularly those in the proximal aspects of the pulmonary arteries, can be quite large requiring a larger retriever catheter and wires than for most DVT cases. Biplane fluoroscopic systems such as those used for cerebral angiography and intervention can be used to improve the catheterization process.

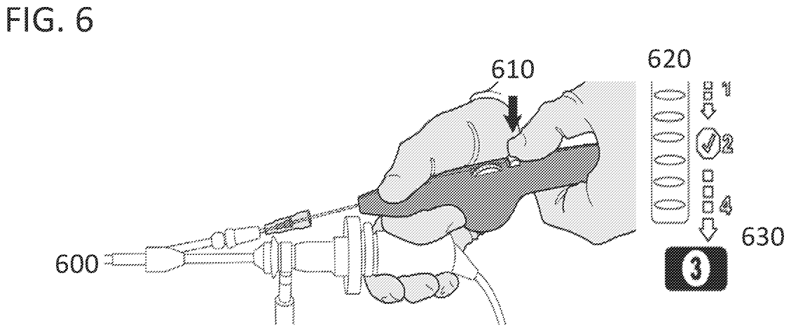

The handle 600 (FIG. 6) may have a proximal end portion and a distal end portion. The distal end portion of the handle which may be connected or attached to the proximal shaft. In some embodiments, an adaptor may facilitate a connection between the handle and the proximal shaft. The handle may be formed from a polymer material, a metal material, a combination of metal material and a polymer material, and/or one or other suitable materials. Further, the handle may be formed with a suitable forming technique including machining, molding, grinding, injection molding, and laser cutting.

In one embodiment shown in FIG. 6, the device has a custom-made handle 600, in which an operator can engage a thumb engaging surface of the device 610 to transmit movement to the device through a compressed coil spring 620, wherein the operator is able to apply a calibrated force 630 to the device. The calibrated forces may be spaced a predetermined linear distance, to include detents, cut-outs, recesses, spacings, notches, indents, bumps, protrusions and/or other features. As shown in FIG. 6, the calibration forces may be linearly drawn as it is moved one or more predetermined distances in the longitudinal direction. In one example, the adjustment member may include a portion having a protrusion to include a cut-out, recess, spacing, notch, indent, and/or other formations to facilitate engaging the restrictions in or on the handle.

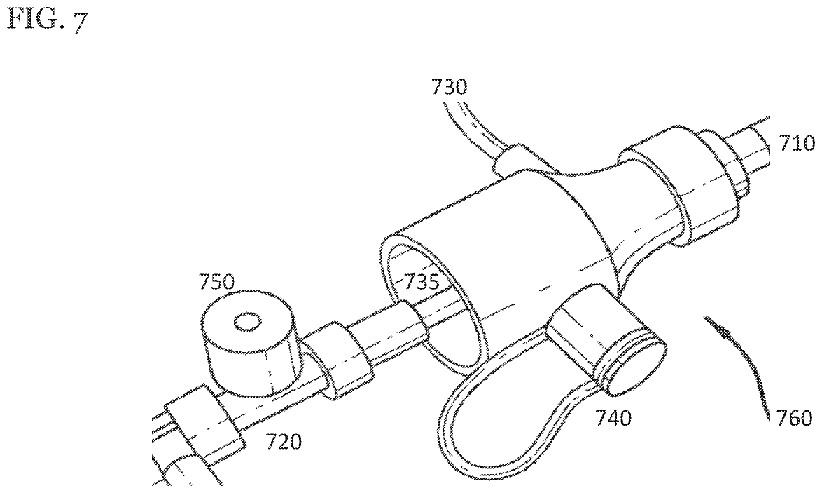

As shown in FIG. 7, a catheter 710 connected to an aspiration device 720 can be added to the manifold attached to the hub of the guiding catheter 730 or the collecting catheter 735 so that aspiration can be applied to the entire system to facilitate thrombus retraction, and prevent fragmentation and embolization of fragments through the guiding catheter 730. Aspiration coupled with mechanical thrombectomy 740 can thereby assist in the retraction of large clots. An aspirational system attached to the manifold of the guiding catheter may help reduce the size of the clot within the collecting device 735 to facilitate removal of the thrombus. Aspiration can also reduce the potential for distal embolization as the clot is manipulated.

The entire thrombus may be pulled into the guiding catheter 3 and removed from the body, leaving the guiding catheter 3 in place. If the clot is too large to be pulled into and through the guiding catheter 3, aspiration may be applied to the guiding catheter or the collecting catheter 4, wherein a catheter connected to an aspiration system can be hooked to the flushing system for the guiding catheter via a 3-way stopcock 750. Aspiration 760 can thus be usefully added when applied to the clot that has been pulled into the collecting device 4 to make it smaller for removal through the guiding catheter 3.

A method of treating deep vein thrombosis and pulmonary embolisms may include accessing a venous vessel of a patient, wherein a retraction catheter containing a clot treatment device is inserted into the venous circulatory system to a site of clot, wherein an aspiration catheter in inserted with wall-mounted suction attached to its inflow port, wherein the aspiration component can remove clot and other debris, and, wherein complete removal of both soft and hard components of a vascular obstruction is completed with one pass within in ninety percent of cases.

A device that may be used in the method may include a device equipped with a collecting mechanism in the form of a collecting catheter that passes over the retraction catheter, and that is equipped with a collecting structure that can be deployed when moving the collecting catheter beyond the end of the guiding catheter, surrounding the object when the object is extracted using the retraction catheter.

The method may further include accessing a venous vessel, inserting into retraction catheter into vessel, and restoring blood flow using the clot retraction device.

An alternative multi-lumen, multi-functional catheter system may include a plurality of axial lumens, wherein at least one physiological measuring device is present within a clot retraction catheter, wherein said physiological measuring device is connected to a host computer which is equipped for receiving information regarding DVT and PE treatment plans, wherein the host computer contains a treatment planning and therapy algorithm for individual DVT and PE patients, and, wherein the host computer signals the operator to actively modify the existing treatment plan as the therapy algorithm progresses.

A thrombectomy catheter comprising: an elongate flexible catheter body having a proximal end, a distal end and a central lumen extending longitudinally through the catheter body, wherein the catheter comprises a catheter with a variable durometer outer jacket, wherein the catheter wall thickness ratio of the inner diameter to the outer diameter is 0.80 or higher, wherein the tensile strength of the catheter is higher than 2 lbs.

Another device for removing blood clots may include an intravascular catheter having a distal end and a proximal end, the catheter having an inner lumen and an outer lumen, wherein an aspiration pump is attached to the proximal end of the catheter, and a mechanically actuated positive displacement powered by a rotating motor, wherein the motor rotates at a speed below 2000 RPM when driving the aspiration pump and wherein the speed of the motor is cycled at a frequency below 10 Hz.

Another method of treating deep vein thrombosis in a peripheral vasculature of a patient may include: percutaneously accessing a venous vessel of a patient with an introducer sheath through an access site into the venous vessel of the patient; inserting a catheter constraining a thrombectomy device through the lumen of the introducer sheath so that a distal tip of the catheter is distally past a portion of the thrombus; deploying the thrombectomy device from a constrained configuration to an expanded configuration, wherein the thrombectomy device is in an expanded state between about 20 degrees and about 50 degrees; and, removing the thrombectomy device from the patient.

It will be apparent to those skilled in the art that various modifications and variations can be made in the disclosed systems and processes. It is intended that the specification and examples be considered as exemplary only.

Thrombus in the vasculature includes a range of morphologies and consistencies. Typically, older thrombus material contains a higher percentage of fibrin, making it less compressible with a harder outer surface that makes it more difficult to ensnare or aspirate than more acute thrombus which is softer. Current mechanical thrombectomy devices may not penetrate the surface of a hard fibrin-rich thrombus or produce sufficient force to grip the thrombus. It can be very difficult to aspirate a hard thrombus without first breaking it into pieces, which could then embolize into distal branches. During thrombectomy, 75-85% of thrombi can be removed using current devices, such as stent-retrievers and aspirators. However, the remaining 15-25% of intravascular thrombus cannot be easily removed by mechanical devices because the thrombus is hard.

CT and fluoroscopy imaging cannot typically identify the composition of intravascular thrombus, which may vary from relatively hard to relatively gel-like and soft. An obstructing thrombus in a blood vessel of the brain can be a medical emergency caused by occlusion of blood vessels to the brain or within the brain. Although an ischemic event can occur anywhere in the vascular system, the carotid artery bifurcation and the origin of the internal carotid artery are the most frequent sites for thrombotic occlusions of cerebral blood vessels.

Methods for imaging thrombus are reviewed in the present disclosure. As used herein, an imaging technology may include, positron-emission tomography, single photon emission computed tomography, magnetic resonance imaging, optical imaging, ultrasound, photoacoustic imaging, computed tomography, or near-infrared fluorescence-imaging.

FIG. 1H is a cross-sectional view of a catheter 10 in an artery adjacent to intravascular thrombus 62. In an exemplary embodiment of the disclosure, an optical fiber 95 is used to measure an optical signal collected from the thrombus 62, which is then output to an MRI-based tracking system 95. The catheter 10 may be advanced to the thrombus 62 to illuminate the thrombus 62, wherein an optical measurement unit 70 enables identification of the optical signal of the thrombus 42 which reflects the content of the thrombus 62. A detector in the MRI system 99 converts the optical signal 42 into an electrical analog signal, and an analog-to-digital conversion circuit 93 digitizes the signal to the MR tracking system 95 for analysis.

In one embodiment of the disclosure shown in FIG. 1I, a schematic of the brain of a patient illustrates a thrombus retraction procedure 100 in which fluoroscopic images 136, 150, 160 may be acquired and stored in memory in a host computer for subsequent retrieval by an MRI-based tracking system 140.

FIG. 2A shows optical reflection curves 210 that summarize in vitro studies to evaluate hard clot 212, gel-like clot 213 and soft clot 214 collected in vitro. The partial thromboplastic time coagulation assay 215 was used in pig blood in glass test_tubes. Thrombi were placed on gauze and photographed 220 based on linear thrombus dimensions. Thrombus was formalin-fixed, embedded in paraffin, and stained with hematoxylin and eosin. Histological sections 225 were photographed based on erythrocyte-rich 226 and platelet-fibrin 227 accumulations, and neutrophil 228 and monocyte 229 deposits.

In one embodiment, contrast agents were used for optical imaging 230, fluorescence 231, luminescence 232 or acousto-optical imaging 233. In one example, silicon containing nanoparticles 234 were used to produce fluorescence and luminescence signal. Other contrast agents can include nanospheres 240, such metal oxide nanoparticles 241, and quantum dots 242. Photoacoustic imaging contrast agents can include photoacoustic imaging-compatible agents 245, such as methylene blue 246, single-walled carbon nanotubes 247, and gold nanoparticles 248.

FIG. 2B illustrates another embodiment in which thrombi were formed from platelet poor plasma in well plates with phosphate-buffered saline. A non-specific binder was added to the thrombi for 30 minutes then the thrombi were washed with PBS prior to fluorescence imaging 250. Thrombi were subsequently washed and then fluorescence imaged again. Thrombus was imaged by adding an imaging agent to a cell culture or tissue culture containing a thrombus to generate a detectable signal within a fluorescence image 260.

In another embodiment, the optical attenuation characteristics of hard thrombus were evaluated compared to gel-like and soft thrombus. The ratio of reflected to incident light intensities of optical attenuation curves 270 was used to determine whether the thrombus is hard or soft.

In another embodiment, the transmission spectrum of light 280 produced characteristic wavelengths due to absorption by oxygenated red blood cells was compared to a hard thrombus and soft or gel-like clots to determine the presence of deoxygenated hemoglobin in the clot.

FIG. 3A summarizes studies in experimental animals. Digital subtraction angiography 300 was used to confirm vascular occlusion in thrombectomy studies. Photographs of retracted thrombotic material were compared with the architecture of the retained clot 310. Clots were placed on gauze and photographed, gross measurements of linear thrombus dimensions were taken, formalin-fixed, then embedded in paraffin and stained with hematoxylin and eosin. As shown in FIG. 3B, histological sections of carotid artery 320 were photographed after thrombus removal 310.
In one embodiment, clot composition was categorized by light microscopy as RBC-dominant 330, fibrin-dominant 331, or mixed 332. Histopathologic analysis included quantitative and qualitative measurements for RBC 340, WBC 341, and fibrin 342. Image analysis software was used to measure quantities of fibrin 350, RBCs 351, and WBCs 352.

FIG. 4A, B shows a modified clot classification system based on imaging and clot morphology. Both HMCAS 421 and BA 422 were significantly associated with the presence of red blood cell-dominant clots 410. In another embodiment, magnetic resonance imaging contrast agents included a chelating agent selected from paramagnetic metal ions 430, such as Gd(III) 431, Dy(III) 432 and Fe(III) 433.

In another embodiment of the present study, neuroimaging indicators 440 were used to distinguish “red thrombi” 441 from “white thrombi” 442. A tracking system (400, 450, 460, 470) for various image/sensing data, e.g., 430, 410 may be addressed by registration transformation and may be implemented by supplemental drug therapy 440 or adjustment of the mechanical thrombectomy 441 procedures.

In another embodiment, stroke resulting from erythrocyte-rich thrombus 451 in the venous system were evaluated after treatment with recombinant tissue plasminogen activator 450.

In another embodiment, fibrin-specific MRI contrast agents 460 were evaluated for identification of thrombus composition 470 to establish the clot size 471 and composition 472 before and following thrombectomy. The computer 491 executing AI code or algorithms or additional functions 481 is also in the communication system of FIG. 4.

FIG. 5B is a schematic of an MRI-based tracking system 500 which can be used in patient 562a stroke studies to investigate clot composition with respect to the age 550, composition 560 and size of a thrombus 570. During formation of a thrombus, the concentration of paramagnetic hemoglobin and methemoglobin within the clot changes resulting in characteristic alterations in fluorescence contrast imaging 510, optical attenuation 520 and thrombus imaging 530. Observation of these MR imaging changes 599 can be clinically useful in evaluating the potential utility of various alternative interventions, such as, for example, drug thrombolytic therapy 540 and mechanical thrombectomy 541 used by the physician 542. Other changes can also be monitored by diagnostic algorithms 580 and AI algorithms 581 by the host computer 590 and other computers 591.

In one embodiment of the present disclosure, pathological changes in thrombi can be evaluated in terms of clot composition determined by various information generating systems 510, 520, 530. Noninvasive imaging can be acquired in acute stroke cases with non-contrast CT or MRI protocols including gradient-recalled echo sequences 570. Comparisons in stroke patients can include acute cerebral occlusion with non-contrast CT or GRE sequences acquired immediately before endovascular thrombectomy using an optical catheter 540, 541 blinded to clinical, angiographic and pathological variables.

A method for visualizing thrombus in an artery includes a wavelength-specific reflector being advanced to traverse the thrombus, wherein the incident light is selectively reflected at the diagnostic wavelength after interacting with the thrombus, wherein passing the optical signal through the thrombus increases an optical attenuation signal compared with a single pass, wherein the host computer analyzes transmitted optical signals, and, wherein the host computer identifies whether the thrombus is hard or soft based on the wavelength signal.

In the method, an optical fiber is adapted to allow light to interact with the thrombus, wherein hard thrombus absorbs less light than thrombus, and, wherein the MRI system can establish the composition of the thrombus based on the optical attenuation of the thrombus.

A device for tracking thrombus in a patient's vasculature may include a measuring device connected to a host computer that can evaluate thrombus retraction, wherein the device is equipped with both optical sensors and imaging sensors, wherein the host computer contains a therapy algorithm for individual patients, and wherein the host computer can actively modify thrombus retraction as the therapy algorithm progresses.

The above device may have the host computer determine the thrombus composition based its optical transmission, and the MRI can be used to evaluate whether thrombus composition reduces its susceptibility to recombinant tissue plasminogen activator. The host computer may determine retraction routes, speed and status of thrombus for individual patients.

A method for tracking thrombus in the vasculature comprising analyzing the intensity of an optical signal from a sensor in a catheter positioned in a blood vessel of a patient may include using an optical signal from the sensor is attenuated by thrombus, wherein an MRI-based host computer tracks the thrombus by analyzing the measured signal attenuation, and, wherein the location of the thrombus is converted by the MRI-bases host computer into MRI coordinates using a registration transformation.

It should be understood that the foregoing and following descriptions are merely illustrative of the present disclosure. Various alternatives and modifications can be devised by those skilled in the art without departing from the scope or spirit of the disclosure.

A further method of treating deep vein thrombosis in a peripheral vasculature of a patient, the method comprising: percutaneously accessing a venous vessel of a patient with an introducer sheath through an access site into the venous vessel of the patient; inserting a catheter constraining a thrombectomy device through the lumen of the introducer sheath so that a distal tip of the catheter is distally past a portion of the thrombus; deploying the thrombectomy device from a constrained configuration to an expanded configuration, wherein the thrombectomy device is in an expanded state between about 20 degrees and about 50 degrees; and, removing the thrombectomy device from the patient.

FIG. 8 shows a mesh web wire system 800 having different protruding elements. protrusions, dots, or gripping elements (804, 806, 808, 810, 812, 814, 816 818) that are on the wire 802, generally facing inward (towards where a clot or a thrombus would be in contact with the wire 802. Different structures for these protruding elements are exemplified separately to show how the different shapes and dimensions and configurations can be selected to provide unique and designed functions with respect to different types of clots, thrombus and debris based on size, texture, rheology and dimensions of the unwanted materials to be captured. Protrusion 804 is a generic and simplest protrusion to form and likely the easiest to manufacture, comprising a truncated spherical dot. Element 806 is a pyramidal element, with a more pointed tip to grasp thrombus with texture and hard surfaces. Element 808 has inwardly sloped side rising to a flat surface to grasp softer clots. Element 810 has inwardly sloped side rising to a concave surface 810a to grasp soft clots and with edges of the concave surface grasping into the clot, yet retaining a large surface area of contact with the particle to be removed. Element 812 is again a relatively generic and simple protrusion to form. Element 814 is shown with outwardly sloped sides 814a which can be used to trap smaller particles as the wire mesh is withdrawn, the sloped sides capturing particles that might even escape the wire mesh. Element 816 is a truncated spherical element, with the cut through the sphere sufficiently low as to again create inwardly sloped surfaces 816a which may provide the small particle capture function described for element 814 above. Element 818 is shown with a textured surface 818a which can assist in grasping clots that might have smoother or more slippery surfaces.

A textured, grooved, irregular surface such as in 818a can be provided on any of the individual structures. Many techniques for forming such surfaces such as embossing, leaching of soluble materials (e.g., soluble polymers, salts, sugars, etc.) in the deposited metal, ceramic, composite or polymeric elements, and the like.

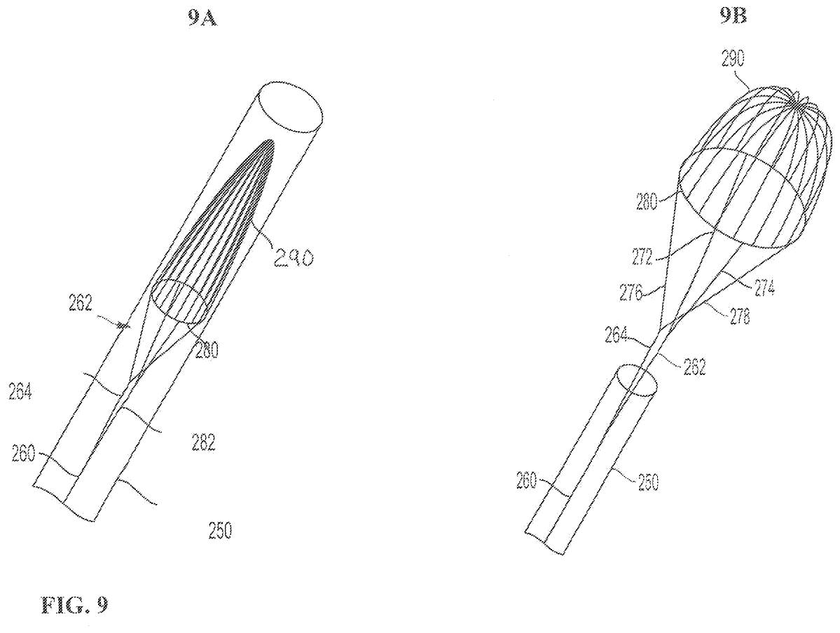

FIG. 9 shows a predeployed 9A catheter 250, forward compressed wire collection element 290 (cross-web wires not shown), compressed ring-shaped element 280, distal guidewires 262 connected to the ring-shaped element 280, primary withdrawal guidewires 264, 282, and the external lead guidewire 260. FIG. 9B shows the deployed system with the distal guidewire set 162 of 9A shown as deployed individual distal guidewires 272, 274, 276, 278.

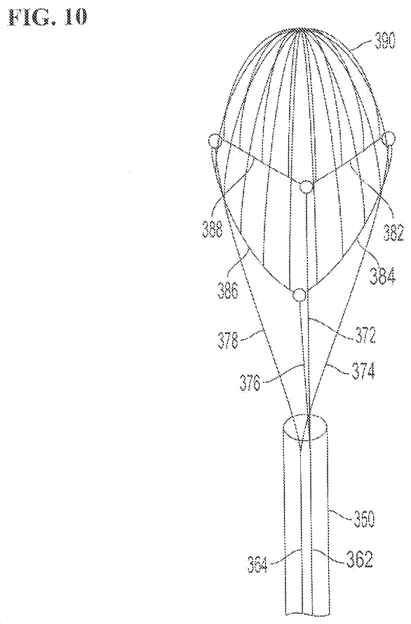

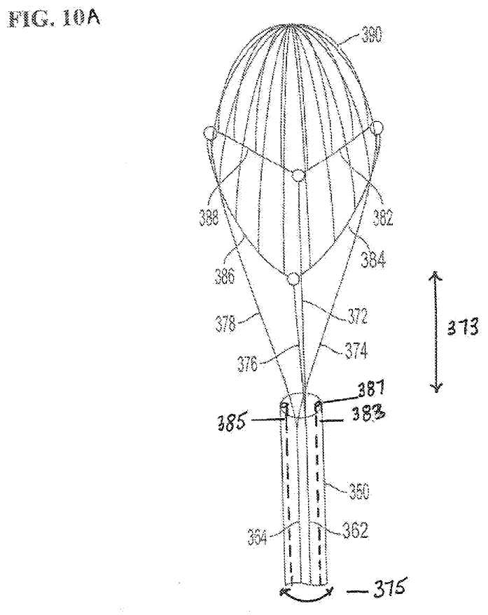

FIG. 10 shows a fully deployed capture system (with the crosswires in the capture web 390 not shown to facilitate other distinguishing elements. The circular ring-shaped element is circular in an aspect view, but from this side view, the connection points for the distal guidewires 372, 374, 376, 378 to the segments of the ring-shaped structure 382, 384, 386, 388 are not within a single plane. The primary retraction guidewires 362, 364 are shown still within the deployment catheter.

FIG. 10A shows the deployed capture system of FIG. 10, but a liner direction 373, rotational direction 375, two sensors 383, 385 and the tip of one sensor 387 identified.

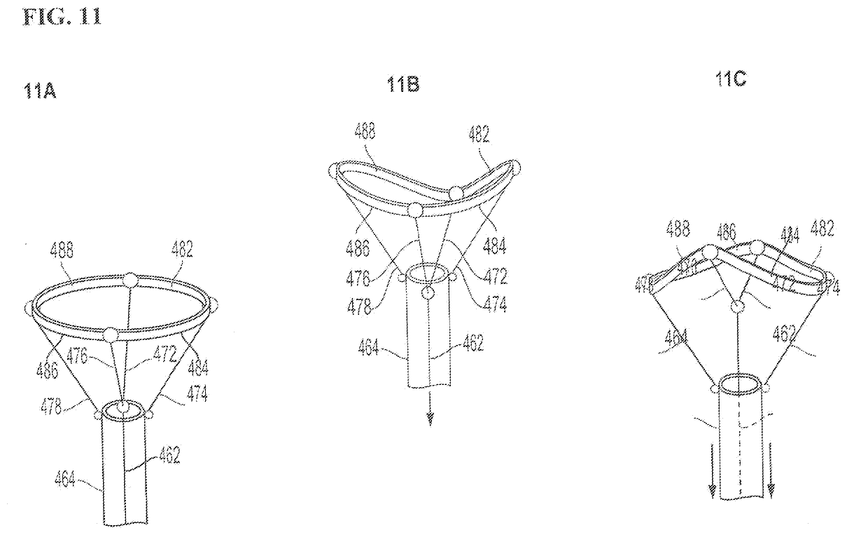

FIG. 11 shows three different aspects or perspectives of the deployed ring-shaped structure's four segments 482, 484, 486, 488 secured by four distal guidewires 472, 474, 476, 478 with a single primary guidewire 462 drawing or releasing the ring-shaped structure and progressively allowing the ring-shaped structure to deform or collapse as progressively shown in FIGS. 11A, 11B and 11C.

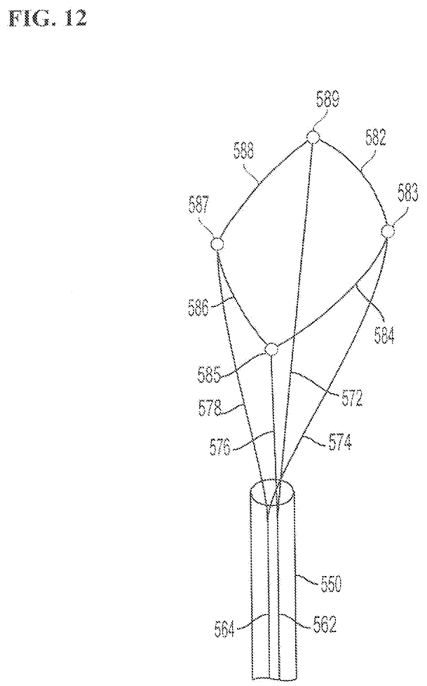

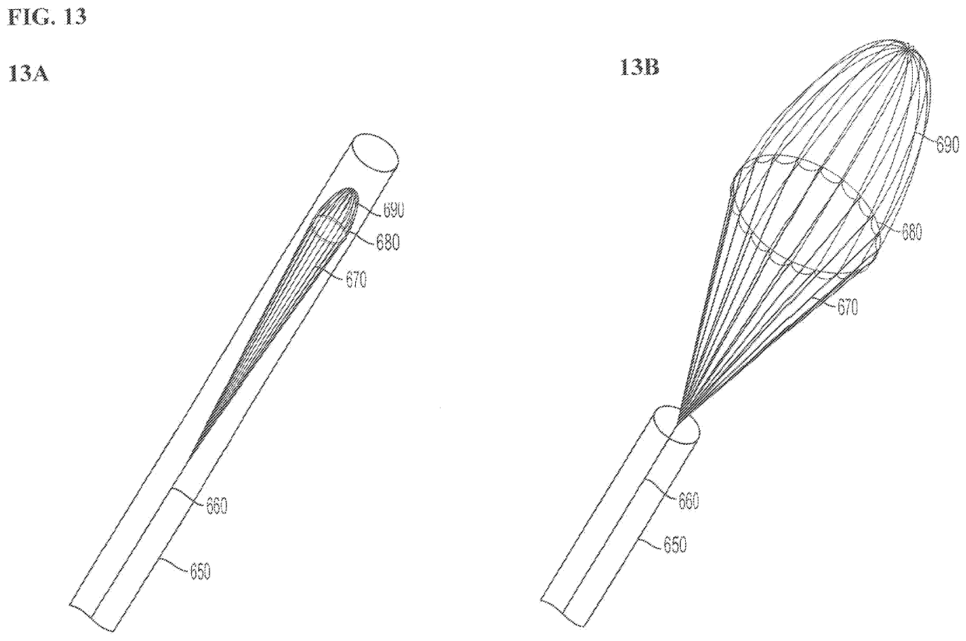

FIG. 12 shows a different perspective of the deployed ring-shaped structure segments 582, 584, 586, 588 with the connection points 583, 585, 587, 589 connecting the segments to the distal guidewires 572, 574, 576, 578 which are in turn connected to primary retraction guidewires 562, 564 within the catheter 550.

FIG. 13 (13A, 13B) shows a pre-deployed 13A and deployed 13B catheter delivery assembly comprising a catheter 650, single retraction guidewire 660, multiple dual wire element distal retraction guidewires 670, the dual wire element distal retraction guidewires 670 being attached to sides of struts 680 forming the ring-shaped circular opening-supporting elements for the wire mesh web 690 (shown without cross wire hatching).

The current invention uses evolutionary programming techniques that often work well because they combine and/or modify code in ways that programmers would not have thought of or chosen to experiment with. Generally, though, evolutionary programming techniques perform with little or no knowledge of or guidance related to the context of the programs that are being combined and/or modified. As such, these techniques lack the ability to make evolutionary programming changes based on those known aspects and may take too long to evolve code in a beneficial manner.

For example, in the practice of the current technology, evolutionary programming techniques may be used to evolve a set of programs that, at least in part, calculate an operational function, such as a sine function. In general, the evolutionary programming technique might combine two programs, but not know where the sine function calculation may be executed (which may be rotational or elevational or extension movement of the components of the thrombectomy devices used in the present invention. As such, the process may evolve the sine function based on a combination of the sine function in one set of code with code that calculates something other than the sine function (e.g., vibration functions, sonic emission function, heat function, visualization enhancement, cryogenic function, drug release function, etc.) from the other set of code. As such, the result is less likely to result in a beneficial outcome (e.g., a better functioning or “higher beneficial procedure” program, by whatever measure might be appropriate). Even if the evolutionary programming technique uses a code-matching algorithm to attempt to find portions of the code that are similar between the two programs, it might fail. For example, even assuming that the code-matching algorithm did find the closest code segments between two programs, depending on the implementation, the code for calculating sine in the first program may actually be more similar to the code for calculating cosine in the second program that it is to the code for calculating sine in the second program. As such, the evolutionary programming technique that used this matching might combine and evolve the code for calculating sine with the code for calculating cosine. Background to this concept that does not effect current novelty can be found in U.S. Ser. No. 11/853,900 (Hazard). All documents reference herein are incorporated in their entirety by reference.

As used herein, “code”, “set of code” or “code set” are broad terms, encompassing numerous embodiments, including, without limitation, full programs (whether compilable, interpretable, executable, or the like), portions of programs, libraries, context-action pairs, data and data structures, and the like.

Mutations of the operational (surgical) codes procedures may be done randomly, or based on “seeding” the system with various parameters. For example, those working on the system, such as programmers, operators, technicians, doctors, nurses, trainers, etc. may know that the angle of a turn or extension of a component of the device should increase and the speed should decrease the closer a capture device element comes to making a procedural step execution or change, but not know which function is correct. So, they may provide seed functions or general constraints, and the system may virtually “experiment” with various functions that use those seed function and/or meet those general constraints. For example, the system may be seeded with various functions or portions of functions for turn angle, for example, the system could be seeded that the turn angle is likely the function of one or more of sine (speed), cos(speed), 1/speed, 1/DTL, speed, DTL, min(0°), max(30°), etc. Then the system could insert one or more of these elements to make functions for the left turn angle. This could be done while taking into account the candidate code (Specific observationally stored information for a specific patient and specific clot), or may be independent of the candidate code.

In some embodiments, the code and therefore program mutations are a resampling of numbers in the context and/or action. For example, the resampling of numbers in the context and/or action may simply be varying the code set numbers using any function, including: sampling within a set percent, sampling the numbers over the observed range of the numbers, or resampling using a maximum entropy distribution with a mean at the number from the original code. As an example of maximum entropy distribution, if a number from the context or action is known to be nonnegative but no other domain knowledge is known about the distribution of that number in other contexts/actions, a resample may consist of drawing a random number from the maximal entropy distribution for a nonnegative number for a given mean, namely an exponential distribution, whose mean is represented by the original number from the context or action. For example, just looking at the sample from Patient One, the distance to the light might be resampled using a maximum entropy distribution with mean of 120 microns, which might result in an attempted or executed movement of 112.5 microns. Further, if the code set has certain observed properties, then the mutated number may be constrained to meet those properties. For example, if observed values are positive, the system may maintain the mutated value as a positive value. If the observed values are integers, the system may maintain the mutated value as an integer. If something out of the typical range of parameters is known about the domain, it can be used in the system to hold the mutations within those known constraints. As such, in some embodiments, the system can allow a domain expert to constrain parts of the context and/or the action. For example, if it is known that the patient has Sjogren's syndrome, then the system can constrain any mutations to being either 0 or 1 (or True or False, depending on the implementation).

In some embodiments, the system may include per-context-field modifiers or constraints. These can be the same or different between code sets. These modifiers might act on the data in the code set. Such actions might be a Get (e.g., clamp), Mutate (e.g., force resampling in a certain range), or Mix (e.g., average for two inputs, return one or the other), or another function or constraint. These modifiers can be useful in instances where one might want to override the default way in which the system operates. Further, modifiers might be useful, for example, when it is desired that the code is set to abide by certain constraints, even if the experts or programmers did not abide by those constraints. One such example is abiding by speed or rotational limits or norms. Modifiers might be used to clamps the speed of the components of the devices. For example, Patient One's's code set may have a modifier that limits speed of forward progression of the device to between 0 and 1.0 mm per second, and Patient Two may have the same constraint, or a different constraint such as clamping speed between 0 and 0.85 mm per second and rotational speed between 0 and 5 degrees per second. Any training value outside those constraints may be clamped back to those values. When the modifiers are the same between two candidate code sets being combined, the system may simply include the modifier unchanged. If they are different, then the modifiers might be mixed or bred in a manner similar to that described the above. For example, the modifier for Patients One and Two linear speed speed might be averaged between −0.05 and 0.9 mm per second or resampled in any other way. Modifiers might also be mutated in manners similar to that described above. In some embodiments, when two code sets are mixed or bred as part of evolving a specific procedure, a portion of each is used, resulting in a “whole” or 100% code. For example, in a particular instance, the system may use 40% of Patient One's code and 60% of Patient Two's code, resulting in a 100% or whole code. In some embodiments, the resulting code may be constructed based on more (or less) than 100% combined. For example, the system may use a combined 110% (70% Patient One and 40% Patient Two), or more, of the candidate code sets. Using more than 100% combined code may be advantageous when the evolutionary aspects of the mutation might remove portions of the context and/or action, remove a link between the context and the action, and/or make part of the context invalid. For example, the mutation might remove the indication of rotational speed from the context. If it turns out that the removed portion of the context is needed for proper performance, it could be useful for there to be a way to reintroduce elements, such as using more than 100% combined of the candidate code sets. Generally, combining more than 100% of two candidate code sets, might be implemented as a Boolean “OR” of the two code sets in order to maintain any pieces that are unique to each code, or possibly 80-100% of the Boolean OR of the two decision trees. Further, in some embodiments, it will be useful to keep all of both sets of each code, notwithstanding that there could be some duplication of context variables.

As discussed elsewhere herein, labels may also be related in a hierarchical relationship. In some embodiments, hierarchical labels can help used to evolve code sets by filling gaps in code where some of the hierarchy of labels is present in the code and other parts of the hierarchy are not. Returning to the mailcart example, if #hardleft and #slowleft are sublabels of #rotateleft, and code has only labels #rotateleft, then the techniques herein may use that information to match or add code to fill out those missing sublabels. For example, if the first candidate code has #leftturn and #slowleft, and the second candidate code has #leftturn and #hardleft, then the compatibility score may be high (and the code must execute a subfile).

U.S. Ser. No. 11/850,057 (Wybo) describes a method of alerting a user to an artificially induced neuromuscular response in a subject includes generating a mechanomyography (MMG) output signal corresponding to a mechanical motion of a muscle of a subject, applying a wavelet transform to the MMG output signal (or in the practice of the present invention, size alteration, vessel surface movement, vibration, etc.) to determine a convolution coefficient for each of a plurality of daughter wavelets, summing the convolution coefficients determined across the plurality of daughter wavelets at each timestep across a plurality of timesteps to generate a net-convolution coefficient (NCC), identifying one or more peaks in the NCC via a peak finding algorithm, and alerting a user of an artificially induced internal response following the identification of one or more peaks in the NCC. Each daughter wavelet of the plurality of daughter wavelets is a time-scaled variant of a common mother wavelet, and the convolution coefficient is indicative of a similarity between the daughter wavelet and the MMG output signal.

During a surgical procedure, the user/surgeon may selectively administer the stimulus to intracorporeal tissue within the treatment area via the desired component of the device to identify the presence of one or more physiological elements in the patient or to test the function of a previously identified physiological element. In some embodiments, the user/surgeon may administer the stimulus via an electrode on a stimulator, for example, upon depressing a button or foot pedal type input device or by tapping a soft-key on the user input display. The electrical stimulus may, for example, be a periodic stimulus that includes a plurality of sequential discrete pulses (e.g., a step pulse) provided at a frequency of less than about 20 Hz, or between about 2 Hz and about 16 Hz. Each pulse may have a pulse width within the range of about 50 μs to about 400 μs. In other examples, each discrete pulse may have a pulse width within the range of about 50 μs to about 200 μs, or within the range of about 75 μs to about 125 μs. Additionally, in some embodiments, the current amplitude of each pulse may be independently controllable.

If a particle breaks off within a predetermined distance of the clot or capture volume of the device, an identified component or element activity (rotation, pronation, extension) may cause the particle to again fall within the capture area, resulting in a mechanical recapture. As noted above, each NMS may be specially configured to monitor a local mechanical movement of an adjacent thrombus/particle group of the subject. In response to this sensed movement, each respective mechanical sensor may generate a mechanomyography (MMG) output signal that corresponds to the sensed mechanical movement, force, and/or response of the adjacent muscle. The MMG output signal may be either a digital or analog signal, and the NMS may further include any communication circuitry or local processing circuitry that may be required to transmit the MMG output signal (or a suitable representation thereof) to the host processor via a wired or wireless communications. In some embodiments, the NMS may further include a local alert capability, such as a lighting module or audible alert module that may operate at the direction of the local processing circuitry or local processor to provide a corresponding visual or audible alert upon the detection of an event.

### Detection Algorithm

As noted above, the system may include resident software, firmware, or embedded processing routines that are operative to analyze the output from the sensors in an effort to identify activities or responses that were induced by an events (i.e., a natural or an induced response). More specifically, these techniques/algorithms may attempt to establish with a high degree of confidence, that a detected movement is a beneficia, neutral or harmful result and that the detected motion is not simply a subject-intended muscle movement, an environmentally caused movement (e.g., bumping the operating table), or an artifact of another aspect of the procedure (e.g., sequential compression devices or cautery). In varying embodiments, the detection techniques/algorithms may be performed in the analog/time domain, the digital/frequency domain, and/or may employ one or more wavelet analyses in an effort to promptly and accurately characterize any sensed motion. Additional techniques such as response gating, stimulus frequency modulation, artificial intelligence/structured machine learning, and/or ensemble approaches may also be used to make this detection more robust and/or provide a greater degree of confidence in the detection. While different detection techniques may each prove to be sufficiently effective in making this characterization, in many instances, however, detection confidence and detection speed/time are in conflict. The following will summarize analog/time domain detection techniques, digital/frequency detection techniques, and then go into further detail on wavelet-style analyses that have been found to generate more rapid responses for comparable levels of accuracy and at higher degrees of confidence.

### Analog/Time Domain Event Detection

In some embodiments, the signal processing algorithms used to recognize a natural or induced response may involve one or more analog detection techniques such as described, for example, in U.S. Pat. No. 8,343,065, issued on Jan. 1, 2013 (the '065 patent), which is incorporated by reference in its entirety. In the analog techniques, the processor may examine one or more aspects of the MMG output signal in an analog/time domain to determine if the sensed response includes signal attributes that are indicative of a response of the muscle to the stimulus. These analog aspects may include, for example, the time derivative of acceleration or the maximum amplitude of the M-wave/initial response being above a predetermined threshold. While these signal traits often have a high degree of sensitivity, they often deliver a significant number of false positives if viewed in isolation (i.e., a single spike in the waveform could just as easily be caused by a sharp bump of the operating table). As such, to provide a robust determination, multiple consecutive events need to be detected to make a final characterization. That said, in many instances ample muscle settling time must be provided between adjacent events to ensure that sequential muscle contractions do not overlap to introduce constructive or destructive signal interference in the waveform parameters, which are often dependent on absolute magnitudes or rates of change. The requirement for muscle settling time could limit the stimulation frequency to less than about 4 Hz, or even 2 Hz or less.

### Digital/Frequency Domain

In a digital context, such as described in US 2015/0051506, filed on Aug. 13, 2013, which is incorporated by reference in its entirety, the processor may convert the analog waveform into the frequency domain (e.g., via a discrete Fourier transform, or fast fourier transform) and then compare the frequency characteristics of the MMG output signal with the known frequency of the applied stimulation to determine whether the sensed muscle responses and/or “events” were induced by the applied stimulus. While this is a more robust form of detection than simply searching for discrete analog signal characteristics, the Fourier transform necessarily requires a certain amount of accumulated data to perform the spectral decomposition. Thus, any performed analysis is necessarily occurring on buffered data and thus is delayed.

### Wavelet Analysis

As a third potential manner of detecting artificially induced environment or even muscle responses, the system may include software or firmware that performs a wavelet similarity analysis on the incoming signals. The use of wavelet signal analyses presents an improvement over the frequency-domain detection techniques as it operates on real-time data as it is received without the need to convert to the frequency domain via an FFT. Likewise, it provides a more robust characterization than simply examining discrete signal parameters (e.g., magnitude or rate of change) in isolation.

In a wavelet analysis, one or more analog wave patterns may be pre-selected as being reference “mother wavelets” that bare a resemblance to a smoothed MMG event. A filtered analog waveform in the MMG output signal may then be compared, in real time, to each mother wavelet to determine a degree of similarity between the two. If the presence of the mother wavelet is found within the analog signal, then the system may infer that an artificially induced muscular event has occurred. This is a more robust analysis than the analog method described above largely because it considers the entire wave shape rather than instantaneous parameters.

Because the responsiveness of each subject's body (and/or tissue groups) may have different dynamic properties, in some embodiments, the system may also search for the presence of different time-scaled variants of the mother wavelet within the analog signal. These variants are generally referred to as “daughter wavelets,” and are similar to the mother wavelet except in how compressed or stretched the wave is on the time-axis.

To perform this analysis, the system may first derive a plurality of “daughter wavelets” from each mother wavelet, where the daughter wavelets are each time-scaled versions of their respective mother wavelet. When analyzing an incoming wave, the examined wave may be continuously passed across each daughter wavelet to determine a respective degree of similarity between the incoming signal and each daughter wavelet (i.e., the degree of similarity being expressed in the form of a “convolution coefficient”). The convolution coefficient for each daughter wavelet may then vary with time as the examined wave passes across the daughter wavelet. This analysis may be performed, for example, using a continuous wavelet transform or discrete wavelet transform and may output a 2d matrix of convolution coefficients such as represented via a heat map. A convolution coefficient may be continuously computed for each scaled daughter wave (represented across a Y/Scale axis) and may be output continuously over time (represented on an X/Time axis. It should be appreciated that other wavelet-based analysis techniques exist (most commonly in the field of digital image compression) and may be used in combination with or instead of continuous or discrete wavelet transforms for the purposes described herein.

U.S. Pat. No. 1,850,332 (Shelton) Methods for treating tissue are provided. In one embodiment, an adjunct material, when secured to tissue, can receive at least one physiological element released from the tissue during healing progression of the tissue, and can exhibit first and second stiffnesses in compression that are approximately constant during first and second time periods from contact with the tissue, with the second stiffness decreasing with time as a function of at least one of oxidation, enzyme-catalyzed hydrolysis, and change of pH resulting from interaction with the at least one physiological element. In another embodiment, the adjunct can receive a unit volume of fluid that causes first and second portions of the adjunct to expand according to first and second expansion behaviors that differ from one another to apply different pressures to the tissue.

Various chemistry including pharmaceuticals and active agents may be introduced during the procedure. Non-limiting examples of antimicrobial agents include Ionic Silver, Aminoglycosides, Streptomycin, Polypeptides, Bacitracin, Triclosan, Tetracyclines, Doxycycline, Minocycline, Demeclocycline, Tetracycline, Oxytetracycline, Chloramphenicol, Nitrofurans, Furazolidone, Nitrofurantoin, Beta-lactams, Penicillins, Amoxicillin, Amoxicillin+Clavulanic Acid, Azlocillin, Flucloxacillin, Ticarcillin, Piperacillin+tazobactam, Tazocin, Biopiper TZ, Zosyn, Carbapenems, Imipenem, Meropenem, Ertapenem, Doripenem, Biapenem, Panipenem/betamipron, Quinolones, Ciprofloxacin, Enoxacin, Gatifloxacin, Gemifloxacin, Levofloxacin, Lomefloxacin, Moxifloxacin, Nalidixic Acid, Norfloxacin, Sulfonamides, Mafenide, Sulfacetamide, Sulfadiazine, Silver Sulfadiazine, Sulfadimethoxine, Sulfamethizole, Sulfamethoxazole, Sulfasalazine, Sulfisoxazole, Bactrim, Prontosil, Ansamycins, Geldanamycin, Herbimycin, Fidaxomicin, Glycopeptides, Teicoplanin, Vancomycin, Telavancin, Dalbavancin, Oritavancin, Lincosamides, Clindamycin, Lincomycin, Lipopeptide, Daptomycin, Macrolides, Azithromycin, Clarithromycin, Erythromycin, Roxithromycin, Telithromycin, Spiramycin, Oxazolidinones, Linezolid, Aminoglycosides, Amikacin, Gentamicin, Kanamycin, Neomycin, Netilmicin, Tobramycin, Paromycin, Paromomycin, Cephalosporins, Ceftobiprole, Ceftolozane, Cefclidine, Flomoxef, Monobactams, Aztreonam, Colistin, and Polymyxin B.

Non-limiting examples of antifungal agents include Triclosan, Polyenes, Amphotericin B, Candicidin, Filipin, Hamycin, Natamycin, Nystatin, Rimocidin, Azoles, Imidazole, Triazole, Thiazole, Allylamines, Amorolfin, Butenafine, Naftifine, Terbinafine, Echinocandins, Anidulafungin, Caspofungin, Micafungin, Ciclopirox, and Benzoic Acid.

Non-limiting examples of antiviral agents include uncoating inhibitors such as, for example, Amantadine, Rimantadine, Pleconaril; reverse transcriptase inhibitors such as, for example, Acyclovir, Lamivudine, Antisenses, Fomivirsen, Morpholinos, Ribozymes, Rifampicin; and virucidals such as, for example, Cyanovirin-N, Griffithsin, Scytovirin, α-Lauroyl-L-arginine ethyl ester (LAE), and Ionic Silver.

Non-limiting examples of anti-inflammatory agents include non-steroidal anti-inflammatory agents (e.g., Salicylates, Aspirin, Diflunisal, Propionic Acid Derivatives, Ibuprofen, Naproxen, Fenoprofen, and Loxoprofen), acetic acid derivatives (e.g., Tolmetin, Sulindac, and Diclofenac), enolic acid derivatives (e.g., Piroxicam, Meloxicam, Droxicam, and Lornoxicam), anthranilic acid derivatives (e.g., Mefenamic Acid, Meclofenamic Acid, and Flufenamic Acid), selective COX-2 inhibitors (e.g., Celecoxib (Celebrex), Parecoxib, Rofecoxib (Vioxx), Sulfonanilides, Nimesulide, and Clonixin), immune selective anti-inflammatory derivatives, corticosteroids (e.g., Dexamethasone), and iNOS inhibitors.

Non-limiting examples of growth factors include those that are cell signaling molecules that stimulate cell growth, healing, remodeling, proliferation, and differentiation. Exemplary growth factors can be short-ranged (paracrine), long ranged (endocrine), or self-stimulating (autocrine). Further examples of the growth factors include growth hormones (e.g., a recombinant growth factor, Nutropin, Humatrope, Genotropin, Norditropin, Saizen, Omnitrope, and a biosynthetic growth factor), Epidermal Growth Factor (EGF) (e.g., inhibitors, Gefitinib, Erlotinib, Afatinib, and Cetuximab), heparin-binding EGF like growth factors (e.g., Epiregulin, Betacellulin, Amphiregulin, and Epigen), Transforming Growth Factor alpha (TGF-a), Neuroregulin 1-4, Fibroblast Growth Factors (FGFs) (e.g., FGF1-2, FGF2, FGF11-14, FGF18, FGF15/19, FGF21, FGF23, FGF7 or Keratinocyte Growth Factor (KGF), FGF10 or KGF2, and Phenytoin), Insuline-like Growth Factors (IGFs) (e.g., IGF-1, IGF-2, and Platelet Derived Growth Factor (PDGF)), Vascular Endothelial Growth Factors (VEGFs) (e.g., inhibitors, Bevacizumab, Ranibizumab, VEGF-A, VEGF-B, VEGF-C, VEGF-D and Becaplermin). Additional non-limiting examples of the growth factors include cytokines, such as Granulocyte Macrophage Colony Stimulating Factors (GM-CSFs) (e.g., inhibitors that inhibit inflammatory responses, and GM-CSF that has been manufactured using recombinant DNA technology and via recombinant yeast-derived sources), Granulocyte Colony Stimulating Factors (G-CSFs) (e.g., Filgrastim, Lenograstim, and Neupogen), Tissue Growth Factor Beta (TGF-B), Leptin, and interleukins (ILs) (e.g., IL-1a, IL-1b, Canakinumab, IL-2, Aldesleukin, Interking, Denileukin Diftitox, IL-3, IL-6, IL-8, IL-10, IL-11, and Oprelvekin). The non-limiting examples of the growth factors further include erythropoietin (e.g., Darbepoetin, Epocept, Dynepo, Epomax, NeoRecormon, Silapo, and Retacrit). Non-limiting examples of analgesics include Narcotics, Opioids, Morphine, Codeine, Oxycodone, Hydrocodone, Buprenorphine, Tramadol, Non-Narcotics, Paracetamol, acetaminophen, NSAIDS, and Flupirtine.

Non-limiting examples of anesthetics include local anesthetics (e.g., Lidocaine, Benzocaine, and Ropivacaine) and general anesthetic. Non-limiting examples of tissue matrix degradation inhibitors that inhibit the action of metalloproteinases (MMPs) and other proteases include MMP inhibitors (e.g., exogenous MMP inhibitors, hydroxamate-based MMP inhibitors, Batimastat (BB-94), Ilomastat (GM6001), Marimastat (BB2516), Thiols, Periostat (Doxycycline), Squaric Acid, BB-1101, Hydroxyureas, Hydrazines, Endogenous, Carbamoylphosphates, Beta Lactams, and tissue Inhibitors of MMPs (TIMPs)). Exemplary medicants also include agents that passively contribute to wound healing such as, for example, nutrients, oxygen expelling agents, amino acids, collageno synthetic agents, Glutamine, Insulin, Butyrate, and Dextran. Exemplary medicants also include anti-adhesion agents, non-limiting examples of which include Hyaluronic acid/Carboxymethyl cellulose (seprafilm), Oxidized Regenerated Cellulose (Interceed), and Icodextrin 4% (Extraneal, Adept). Exemplary medicants also include agents that encourage blood supply regeneration following coronary artery disease (CAD) (e.g., VEGF₁₆₅ protein, AdVEGF.sub.165, AdVEGF₁₂₁, and VEGF₁₆₅ plasmid) or periphery artery disease (PAD) (e.g., VEGF₁₆₅ plasmid, AdVEGF₁₂₁, SB-509 (SFP-VEGF plasmid), AdVEGF₁₆₅, and Ad2-HIF1α-VP16 (WALK trial)).

### Drug Release

An adjunct (optional functional procedural element or component) in accordance with the described techniques can be associated with at least one medicant in a number of different ways, so as to provide a desired effect, such as on tissue in-growth, in a desired manner. The at least one medicant can be configured to be released from the adjunct in multiple spatial and temporal patterns to trigger a desired healing process at a treatment site. The medicant can be disposed within, bonded to, incorporated within, dispersed within, or otherwise associated with the adjunct. For example, the adjunct can have one or more regions releasably retaining therein one or more different medicants. The regions can be distinct reservoirs of various sizes and shapes and retaining medicants therein in various ways, or other distinct or continuous regions within the adjuncts. In some aspects, a specific configuration of the adjunct allows it to releasably retain therein a medicant or more than one different medicant.

Regardless of the way in which the medicant is disposed within the adjunct, an effective amount of the at least one medicant can be encapsulated within a vessel, such as a pellet which can be in the form of microcapsules, microbeads, or any other vessel. The vessels can be formed from a bioabsorbable polymer. Targeted delivery and release of at least one medicant from an adjunct can be accomplished in a number of ways which depend on various factors. In general, the at least one medicant can be released from the adjunct material as a bolus dose such that the medicant is released substantially immediately upon delivery of the adjunct material to tissue. Alternatively, the at least one medicant can be released from the adjunct over a certain duration of time, which can be minutes, hours, days, or more. A rate of the timed release and an amount of the medicant being released can depend on various factors, such as a degradation rate of a region from which the medicant is being released, a degradation rate of one or more coatings or other structures used to retains the medicant within the adjuncts, environmental conditions at a treatment site, and various other factors. In some aspects, when the adjunct has more than one medicant disposed therein, a bolus dose release of a first medicant can regulate a release of a second medicant that commences release after the first medicant is released. The adjunct can include multiple medicants, each of which can affect the release of one or more other medicants in any suitable way. Release of at least one medicant as a bolus dose or as a timed release can occur or begin either substantially immediately upon delivery of the adjunct material to tissue, or it can be delayed until a predetermined time. The delay can depend on a structure and properties of the adjunct or one or more of its regions.

An adjunct material can be configured to have a structure that facilitates distribution of effective amounts of one or more medicants carried within the adjunct to provide a desired effect. For example, the targeted delivery of the medicants can be accomplished by incorporating the medicants into regions (e.g., reservoirs such as pores or other structures) within the adjunct formed in a pattern that allows a certain spatial distribution of the medicants upon their delivery. The medicants disposed within the reservoir can be incorporated into distinct vessels. A reservoir can include more than one type of different medicants. The one or more medicants can be eluted from the adjunct in a homogeneous manner or in heterogeneous spatial and/or temporal manner to deliver a desired therapy. The structure of the adjunct and the way in which the medicants are released therefrom can be used to influence or control tissue re-growth. Moreover, the tissue regrowth can be encouraged in certain locations at the treatment site and discouraged at other locations at the treatment site.

### Fluid Control Features and Drug Release Features

In certain embodiments, the adjuncts (required or optional components providing specific functions beyond only linear movement of the thrombectomy device) can have a variety of configurations that are designed to control fluid (typically only liquid or gel) movement into, out of, and/or through the adjuncts when the adjuncts are in a deployed state (e.g., adjacent to vessels and thrombi in vivo). This fluid control can encourage the mobility of cells and bi-products into and out of the adjunct during tissue remodeling while the adjunct is in a tissue deployed state. Further, this fluid control can impact the ion level of the tissue that is stapled to the adjunct such that the fluid movement through the adjunct can disrupt or enhance environment effects on tissue remodeling.

U.S. Ser. No. 11/850,104 describes an imaging system (a visualization system) particularly useful in the practice of the present technology. The visualization system includes an imaging display system, and a surgical hub. The visualization system includes an illumination source and an imaging device. The surgical hub includes a processor and a memory device configured to store instructions. The instructions direct the surgical hub processor to control the illumination source to illuminate a surgical site and to control the imaging device to receive the imaging data. The surgical processor processes the imaging data to create display data that is transmitted over a cloud-based network to the imaging display system for display.

The surgical system includes a surgical visualization system and a control circuit configured to propose a portion of an organ to resect based on visualization data from the surgical visualization system, determine a first value of a non-visualization parameter of the organ prior to resection of the portion, and determine a second value of the non-visualization parameter of the organ after resection of the portion. Resection of the portion is configured to yield an estimated capacity reduction of the organ.

In another general aspect, a surgical system for use in a surgical procedure is disclosed. The surgical system includes a surgical visualization system and a control circuit configured to receive an input from a user indicative of a portion of an organ to resect estimate a capacity reduction of the organ from removing the portion based on visualization data from the surgical visualization system. In yet another general aspect, a surgical system for use in a surgical procedure is disclosed. The surgical system includes a surgical visualization system and a control circuit configured to receive first visualization data from the surgical visualization system in a first state of an organ (or in the case of the present technology, the novel consideration of the state of the thrombus), determine a first value of a non-visualization parameter of the organ/thrombus/vessel in the first state, receive second visualization data from the surgical visualization system in a second state, determine a second value of the non-visualization parameter of the organ/thrombus/vessel in the second state, and detect an abnormality based on the first visualization data, the second visualization data, the first value of a non-visualization parameter, and the second value of the non-visualization parameter. In various embodiments, an automated surgical system comprising a visualization system, an imaging display system, and a surgical hub is disclosed. The visualization system typically comprises at least one illumination source and at least one imaging device. The imaging display system comprises a display device in data communication with a cloud-based surgical network. The display device is located remotely from the visualization system and configured to present display data to a user monitoring a surgical procedure. The surgical hub comprises a processor, a memory device, a visualization system interface, and a network communication interface. The memory device comprises instructions that, when executed by the processor, cause the processor to transmit illumination control data to the at least one illumination source via the visualization system interface, receive imaging data from the at least one imaging device via the visualization system interface, process the imaging data to calculate the display data, and transmit the display data to the imaging display system over the cloud-based surgical network via the network communication interface.

Anastomosis is the surgical connection of two tube-like structures inside the body. The AI procedures of the present invention can be concurrently used to indicate and then execute repair of damage to veins or arteries, create a bypass for blocked arteries, connect a donor organ to a blood supply, or rejoin two sections of the intestine after removal of diseased tissue.

In certain instances, a surgical system that incorporates a surgical visualization platform may enable smart dissection in order to identify and avoid critical structures. Critical structures include anatomical structures such as a ureter, an artery such as a superior mesenteric artery, a vein such as a portal vein, a nerve such as a phrenic nerve, and/or a tumor, among other anatomical structures, including the thrombus and migrant clot particles. In other instances, a critical structure can be a foreign structure in the anatomical field, such as a surgical device, surgical fastener, clip, tack, bougie, band, and/or plate, for example. Critical structures can be determined on a patient-by-patient and/or a procedure-by-procedure basis. Example critical structures are further described herein. Smart dissection technology may provide improved intraoperative guidance for dissection and/or can enable smarter decisions with critical anatomy detection and avoidance technology, for example.

A surgical system incorporating a surgical visualization platform may also enable smart anastomosis technologies that provide more consistent anastomoses at optimal location(s) with improved workflow. Cancer localization technologies may also be improved with the various surgical visualization platforms and procedures described herein. For example, cancer localization technologies can identify and track a clot location, orientation, and its margins. In certain instances, the thrombus localizations technologies may compensate for movement of a tool, a patient, and/or the patient's anatomy during a surgical procedure in order to provide guidance back to the point of interest for the clinician.

In certain aspects of the U.S. Ser. No. 11/850,104 disclosure, a surgical visualization platform may provide improved tissue characterization and/or lymph node diagnostics and mapping. For example, tissue characterization technologies may characterize tissue type and health without the need for physical haptics, especially when dissecting and/or placing stapling devices within the tissue. Certain tissue characterization technologies described herein may be utilized without ionizing radiation and/or contrast agents. With respect to lymph node diagnostics and mapping, a surgical visualization platform may preoperatively locate, map, and ideally diagnose the lymph system and/or lymph nodes involved in cancerous diagnosis and staging, for example.

During a surgical procedure, the information available to the clinician via the virtual “naked eye” and/or an imaging system may provide an incomplete view of the surgical site. For example, certain structures, such as structures embedded or buried within an organ or behind a segment of the device, can be at least partially concealed or hidden from view. Additionally, certain dimensions and/or relative distances can be difficult to ascertain with existing sensor systems and/or difficult for the “naked eye” to perceive. Moreover, certain structures can move preoperatively (e.g. before a surgical procedure but after a preoperative scan) and/or intraoperatively. In such instances, the clinician can be unable to accurately determine the location of a critical structure intraoperatively.

When the position of a critical structure is uncertain and/or when the proximity between the critical structure and a surgical tool is unknown, a clinician's decision-making process can be inhibited. For example, a clinician may avoid certain areas in order to avoid inadvertent dissection of a critical structure; however, the avoided area may be unnecessarily large and/or at least partially misplaced. Due to uncertainty and/or overly/excessive exercises in caution, the clinician may not access certain desired regions. For example, excess caution may cause a clinician to leave a portion of a tumor and/or other undesirable tissue in an effort to avoid a critical structure even if the critical structure is not in the particular area and/or would not be negatively impacted by the clinician working in that particular area. In certain instances, surgical results can be improved with increased knowledge and/or certainty, which can allow a surgeon to be more accurate and, in certain instances, less conservative/more aggressive with respect to particular anatomical areas.

In various aspects, the U.S. Ser. No. 11/850,104 disclosure provides a surgical visualization system for intraoperative identification and avoidance of critical structures. In one aspect, the present disclosure provides a surgical visualization system that enables enhanced intraoperative decision making and improved surgical outcomes. In various aspects, the disclosed surgical visualization system provides advanced visualization capabilities beyond what a clinician sees with the “naked eye” and/or beyond what an imaging system can recognize and/or convey to the clinician. The various surgical visualization systems can augment and enhance what a clinician is able to know prior to tissue treatment (e.g. dissection) and, thus, may improve outcomes in various instances.

For example, a visualization system can include a first light emitter configured to emit a plurality of spectral waves, a second light emitter configured to emit a light pattern, and one or more receivers, or sensors, configured to detect visible light, molecular responses to the spectral waves (spectral imaging), and/or the light pattern. It should be noted that throughout the following disclosure, any reference to “light,” unless specifically in reference to visible light, can include electromagnetic radiation (EMR) or photons in the visible and/or non-visible portions of the EMR wavelength spectrum. The surgical visualization system can also include an imaging system and a control circuit in signal communication with the receiver(s) and the imaging system. Based on output from the receiver(s), the control circuit can determine a geometric surface map, i.e. three-dimensional surface topography, of the visible surfaces at the surgical site and one or more distances with respect to the surgical site. In certain instances, the control circuit can determine one more distances to an at least partially concealed structure. Moreover, the imaging system can convey the geometric surface map and the one or more distances to a clinician. In such instances, an augmented view of the surgical site provided to the clinician can provide a representation of the concealed structure within the relevant context of the surgical site. For example, the imaging system can virtually augment the concealed structure on the geometric surface map of the concealing and/or obstructing tissue similar to a line drawn on the ground to indicate a utility line below the surface. Additionally or alternatively, the imaging system can convey the proximity of one or more surgical tools to the visible and obstructing tissue and/or to the at least partially concealed structure and/or the depth of the concealed structure below the visible surface of the obstructing tissue.

For example, the visualization system can determine a distance with respect to the augmented line on the surface of the visible tissue and convey the distance to the imaging system.

In various further aspects of the U.S. Ser. No. 11/850,104 disclosure, a surgical visualization system is disclosed for intraoperative identification and avoidance of critical structures. Such a surgical visualization system can provide valuable information to a clinician during a surgical procedure. As a result, the clinician can confidently maintain momentum throughout the surgical procedure knowing that the surgical visualization system is tracking a critical structure such as a ureter, specific nerves, and/or critical blood vessels, for example, which may be approached during dissection, for example. In one aspect, the surgical visualization system can provide an indication to the clinician in sufficient time for the clinician to pause and/or slow down the surgical procedure and evaluate the proximity to the critical structure to prevent inadvertent damage thereto. The surgical visualization system can provide an ideal, optimized, and/or customizable amount of information to the clinician to allow the clinician to move confidently and/or quickly through tissue while avoiding inadvertent damage to healthy tissue and/or critical structure(s) and, thus, to minimize the risk of harm resulting from the surgical procedure.

U.S. Pat. No. 11,854,690 (Oko) describes an indexing system useful in storing past and current information, useful in executing code for a heuristic analysis and suggestion for current or altered surgical procedures.

U.S. Pat. No. 11,854,193 (Kim) describes an AI approach to verification and confirmation of biopsies and other indicators of systems for cancer indications. The operation of that system may be translated into near real-life identification of properties and changes of properties in thrombi that can be upgraded regularly to provide further information bases for modifying an ongoing thrombectomy procedure.

According to an aspect of the U.S. Ser. No. 11/854,193 disclosure, an apparatus for evaluating validity of detection of a cancer region may be provided. The apparatus may comprise a parametric magnetic resonance imaging (MRI) provider configured to provide at least one MRI constructed based on different parameters, a first cancer region input unit configured to receive a first cancer region based on the at least one parametric MRI, a cancer region processor including a cancer region detection model for receiving the at least one parametric MRI as input and outputting cancer region information and configured to generate and provide guide information corresponding to an image to be analyzed through the cancer region detection model, a second cancer region input unit configured to receive a second cancer region based on the guide information, and a validity evaluator configured to generate validity evaluation information of the second cancer region, by comparing the first cancer region with the second cancer region based on a pathology image obtained by mapping a region, in which cancer is present, of an extracted body portion.

According to another aspect of the U.S. Ser. No. 11/854,193 disclosure, method of evaluating validity of detection of an anomalous region may be provided. The method may comprise providing at least one parametric magnetic resonance imaging (MRI) constructed based on different parameters, receiving a first region based on the at least one parametric MRI, building a region detection model for receiving the at least one parametric MRI as input and outputting region information, providing guide information corresponding to an image to be analyzed through the ambiguous or anomalous region detection model, receiving a second region based on the guide information, and generating validity evaluation information of the second region by comparing the first cancer region with the second region based on a pathology image obtained by mapping a region, in which an anomaly is present, of an examined body portion.

U.S. Pat. No. 8,753,304 (Dacey) discloses systems, devices, methods, and compositions are described for providing an actively-controllable disinfecting implantable device configured to, for example, treat or prevent an infection in a biological subject. The system of the present technology can be overlain and combined with the technology and device of Dacey to adjust operational procedures.

Standard monitoring devices (not shown) have alarm systems built into them. In the practice of the present invention, one may to have the AI connected to existing alarm systems to provide the signal or urgency. Similarly, there are typically alphanumeric displays in the operating room. The AI can provide information, optional procedures, probabilities and other information or communication on the screen. Medical personnel could input requests, directions (if the thrombectomy system were robotic), ask for clarification of AI provided information, add information to the AI to advance its analysis of the status and prognosis for variously altered procedures directed by the AI.

Additional addressing or carried to the region of the surgical procedure would include vibration sensors, particularly those than can be functionally proximal (at a distance at which they are receiving accurate data of vibrations) to the vessels where the procedure is being performed. There are two types of vibrations that can be critical to identify and act upon. One is vibrations caused by the movement of the device and its components dragging or sliding against vessel walls. There will always be some necessary contact that results in vibrations (sensed by actual vibrations or wave patterns in the blood stream), but when the device or its components erratically catch, release vessel interiors, or drag an particle along the vessel walls, there can be incident damage. By identifying this adverse event during surgery, AI can suggest specific adjustments in the procedure including but not limited to one or more of altering directional (typically linear) movement of the device, directional speed, rotation of the individual components or entire device, partial withdrawal or partial advance, aspiration, sonic or chemical shrinking of dangerous size particles, replacement of components in the procedure, or even temporary pausing or complete stoppage of the procedure.

Where undesirable “squishing” and or shape distortion of the particles is sensed and AI interprets a need to address those observed events, a specific response may be provided or executed, or a range of responses (such as those with respect to vibration, and further including sonic and/or thermal and/or chemical provision to the undesirable shape. The provision of available procedures to sensed abnormal dimensional changes can relieve the stress of medical operators and improve overall statistical results in the thrombectomy procedure. Of particular interest is where forces derived from the device alter the dimensions of any section of a clot by more than 5%, more than 7.5%, and especially more than 10%, indicating that the forces are likely to cause a breakup of the clot at that specific location. Exceeding or reaching any of these dimension changing parameters is likely to trigger some measure of alarm. Similarly, any observed breaking-off of a clot piece will trigger an alarm.

The alternative options provided for use by operating or supervising surgery personnel are maintained in or accessed by an AI programmed device on site. The local device may access a distal table of options rather than having the massive information storage capacity needed kept on site. The distal storage also minimizes local breakdowns or power outages, where energy backup storage would likely be directed to critical life support systems.

The surgical process for thrombectomy, using manual or robotic surgical components involves analyzing a component's function and performance requirements, determining its performance during real-time (that is within a time frame where altering results based on sensor observation can occur within a time frame where continuing surgical performance may be altered in an effective time frame to respond to sensed information. The AI data bank can then selection from among options for altering or maintaining the existing procedures to address observations requiring consideration or attention to observed abnormal or out-of-tolerance details.

Conceptual Design. The available procedural collection or design process typically starts with conceptual collection of all operationally available movements and ancillary functional elements to the underlying device, where the basic requirements and constraints of the components and devices and potentially sensed or otherwise observed conditions are defined.

Detailed Performance Design. Conceptual design is followed by detailed design, where the component has defined ranges of operation and performance capabilities designed to meet the requirements and constraints of operating conditions, considering factors such as specific patient information, device and component materials, operation methods, and observed conditions.

Analysis and Optimization (CAE). The available procedures within the operational capabilities of the actual equipment goes through an analysis and optimization stage prior to formation of a collection of available procedures before inclusion in storage. Analysis and optimization rely on computer-aided design (CAD) and 3D mechanical engineering simulation (CAE) tools to ensure the procedures will have a high degree of functioning as intended and meet performance requirements.

Multi-Objective Optimization. Optimizing an operational procedure product means considering several objectives under sensed existing conditions (sometimes contrasting) and constraints coming from targets on the status and condition of the patient and the region of operation for the procedure and size and rheology and properties of the thrombus. This is called multi-objective optimization.

1. A method of alerting a user to the existence of an anomalous variation in local conditions during insertion, deployment, manipulation or withdrawal of a thrombus retrieval device in a subject, the method comprising: inserting a thrombus retrieval device into a blood vessel in a patient having a thrombus within the vessel while a region surrounding and including the thrombus is under sensed observation, sensing an output signal or visualization responsive to at least motion of or within or on the outside of the thrombus, the motion being in response to the insertion, deployment, manipulation or withdrawal of the thrombus retrieval device, sensing the existence of the anomalous variation in the local conditions, a server receiving data of the sensed anomalous variation in local conditions; the server executing software to identify available procedures to be used after identification of one or more anomalous variations in local conditions related to the sensed anomalous variation in local conditions; the server providing first visual data visible to a human eye identifying the available procedures, with or without a hierarchal preference among the available procedures; a medical practitioner manually executing at least one of the identified medical procedures or directing a robotic element to perform at least a portion of the identified medical procedure; and while undergoing continued sensed observation, additional sensed output is transmitted to the server and effects of the execution of the available procedure are compared from the continued sensed observation to evaluate resultant effects on the anomalous variation in local procedures to determine what alteration has occurred in the anomalous variation in local conditions, identifying operational value in the execution of the execution of the available procedure; and the server providing a second provided visual data.

2. The method of claim 1 wherein the anomalous variation in local conditions is selected from the group consisting of changes in shape of the thrombus, particles separating from the thrombus, vibrations from movement of the thrombectomy device, and alterations in the surface of the blood vessel.

3. The method of claim 1 wherein an available procedure to be executed is selected from the group consisting of rotation of a portion of the thrombectomy device, at least partial retraction or extension of the thrombectomy device, alteration in the shape of deployment of a thrombus capture segment of the thrombectomy device, vibrating a distal element in the thrombus capture segment of the thrombectomy device, generating heat or reducing temperature around the thrombus capture segment of the thrombectomy device, introducing chemicals or pharmaceuticals around the thrombus capture segment of the thrombectomy device, and aspirating blood around the thrombus capture segment of the thrombectomy device to withdrawn particles.

4. The method of claim 2 wherein an available procedure to be executed is selected from the group consisting of rotation of a portion of the thrombectomy device, at least partial retraction or extension of the thrombectomy device, alteration in the shape of deployment of a thrombus capture segment of the thrombectomy device, vibrating a distal element in the thrombus capture segment of the thrombectomy device, generating heat or reducing temperature around the thrombus capture segment of the thrombectomy device, introducing chemicals or pharmaceuticals around the thrombus capture segment of the thrombectomy device, providing liquid to the regions, and aspirating blood around the thrombus capture segment of the thrombectomy device to withdrawn particles.

5. The method of claim 1 wherein sensed observation is selected from the group consisting of optical fiber or semiconductor observation, MRI observation or sonogram observation of the region.

6. The method of claim 2 wherein sensed observation is selected from the group consisting of optical fiber or semiconductor observation, MRI observation or sonogram observation of the region.

7. The method of claim 3 wherein sensed observation is selected from the group consisting of optical fiber or semiconductor observation, MRI observation or sonogram observation of the region.

8. The method of claim 4 wherein sensed observation is selected from the group consisting of optical fiber or semiconductor observation, MRI observation or sonogram observation of the region.

9. The method of claim 1 wherein the server applies a wavelet transform to the sensed observation data further includes computing a convolution coefficient for each of a plurality of daughter wavelets, wherein each wavelet of the plurality of daughter wavelets is a time-scaled variant of a common mother wavelet, and wherein the sensed observation data is indicative of a similarity between the wavelet and the sensed observation data signal.

10. The method of claim 1 further comprising providing an indication of statistical confidence for the determination that the anomalous variation in local conditions includes a shape change in the thrombus indicative of internal stress in the thrombus that will break the thrombus into smaller particles in response to the insertion, deployment, manipulation or withdrawal of the thrombus retrieval device.

11. The method of claim 3 further comprising providing an indication of statistical confidence for the determination that the anomalous variation in local conditions includes a shape change in the thrombus indicative of internal stress in the thrombus that will break the thrombus into smaller particles in response to the insertion, deployment, manipulation or withdrawal of the thrombus retrieval device.

12. The method of claim 4 further comprising providing an indication of statistical confidence for the determination that the anomalous variation in local conditions includes a shape change in the thrombus indicative of internal stress in the thrombus that will break the thrombus into smaller particles in response to the insertion, deployment, manipulation or withdrawal of the thrombus retrieval device.

13. The method of claim 5 further comprising providing an indication of statistical confidence for the determination that the anomalous variation in local conditions includes a shape change in the thrombus indicative of internal stress in the thrombus that will break the thrombus into smaller particles in response to the insertion, deployment, manipulation or withdrawal of the thrombus retrieval device.

14. The method of claim 8 further comprising providing an indication of statistical confidence for the determination that the anomalous variation in local conditions includes a shape change in the thrombus indicative of internal stress in the thrombus that will break the thrombus into smaller particles in response to the insertion, deployment, manipulation or withdrawal of the thrombus retrieval device.

15. The method of claim 9 further comprising providing an indication of statistical confidence for the determination that the anomalous variation in local conditions includes a shape change in the thrombus indicative of internal stress in the thrombus that will break the thrombus into smaller particles in response to the insertion, deployment, manipulation or withdrawal of the thrombus retrieval device.

16. The method of claim 10, further comprising providing a control signal to a robotic surgical system if it is determined that the physical motion of the clot is an artificially induced response to movement of the thrombus retrieval device.
